(19) United States
(12) Patent Application Publication
(10) Pub. No.: US 2026/0120198 A1
(43) Pub. Date: Apr. 30, 2026

(54) AUTONOMOUS MEDICAL CLAIM EDIT SYSTEM

(71) Applicant: Oracle America, Inc., Austin, TX (US)

(72) Inventors: Deana Holznecht, Los Angeles, CA (US); Geneva Haines, Kansas City, MO (US); Naresh Revanuru, Redwood City, CA (US); Lijun Wang, Belmont, CA (US); Amritbani Sondhi, Malvern, PA (US); Sanket Korgaonkar, Kansas City, MO (US); Raman Kahlon, Belmont, CA (US)

(73) Assignee: Oracle America, Inc., Austin, TX (US)

(21) Appl. No.: 19/362,880

(22) Filed: Oct. 20, 2025

Related U.S. Application Data
(60) Provisional application No. 63/712,225, filed on Oct. 25, 2024.

(51) Int. Cl. G06Q 40/08; G06Q 10/10; G16H 10/60

(52) U.S. Cl. CPC G06Q 40/09; G06Q 10/10; G16H 10/60

(57) ABSTRACT

Techniques for an autonomous edit process for medical claims are disclosed. An electronic claim associated with a patient encounter is retrieved, along with a flag indicative of the claim being erroneous, and an error report identifying an error condition within the claim. A plurality of heterogeneous electronic medical records associated with the patient encounter is retrieved, the plurality including structured billing codes, structured data, semi-structured data, and/or free-text clinical notes. A feature-extraction engine transforms the plurality of heterogeneous electronic medical records into a unified machine-readable representation including semantic embeddings, which are processed by a trained machine learning (ML) model, to generate a mapping between the error condition and one or more spans within the unified representation. The ML model identifies documentary evidence within the one or more spans that satisfies a model-learned evidentiary relevance condition, and generates one or more machine-formatted corrective actions to resolve the error condition.

### CROSS-REFERENCE TO RELATED APPLICATIONS

This application claims the benefit of U.S. Provisional Patent Application No. 63/712,225 filed on Oct. 25, 2024. The entire disclosure of the aforementioned application is incorporated by reference herein in its entirety for all purposes.

### BACKGROUND

Medical coding and billing are an integral part of a healthcare ecosystem, e.g., for receiving reimbursement for medical costs. For example, after or during a patient encounter with a healthcare professional, charges are generated based on clinical activity and documents indicative of the patient visit. The charges include medical record documentation, such as transcription of clinical notes or after-visit summary captured by medical care professionals, laboratory and radiologic results, list and/or invoice of medical equipment used for a medical procedure, surgical notes, etc. A medical coder then assigns one or more medical codes for the patient encounter. Medical coding is a process by which healthcare diagnosis, procedures, medical services, and equipment are transformed or mapped into universal medical alphanumeric codes. Such medical codes are assigned based on medical record documentation, such as transcription of notes or after-visit summary captured by medical care professionals, clinical notes, surgical notes, laboratory and radiologic results, etc. The medical codes are then used to generate a medical claim (also referred to herein as a claim). The medical claim is submitted to medical insurance carriers for reimbursement.

Errors may occur at any stage in the medical coding and billing process. For example, there may be errors within the charges and medical documents associated with the patient encounter, errors associated with the assigned codes, errors occurring during generation of the claims, etc. Such errors are eventually reflected within the claims, which may delay the reimbursement process from the medical insurance carriers.

### BRIEF SUMMARY

In various embodiments, a computer-implemented method comprises: receiving, by a computing system, (i) an electronic claim associated with a patient encounter, (ii) a flag indicative of the claim being erroneous, and (iii) a corresponding error report identifying an error condition within the claim; retrieving, by the computing system, a plurality of heterogeneous electronic medical records associated with the patient encounter, the plurality including one or more of structured billing codes, structured data, semi-structured data, and free-text clinical notes; transforming, by a feature extraction engine, the plurality of heterogeneous electronic medical records into a unified machine-readable representation comprising semantic embeddings derived from one or more of the structured data, the semi-structured data, and the free-text clinical notes; processing, by a trained machine learning (ML) model, the unified representation and the error condition to: generate a mapping between the error condition and one or more spans within the unified representation, the mapping determined using (i) learned associations between claim error types and record modalities and/or (ii) the semantic embeddings, and identify documentary evidence within the one or more spans that satisfies a model-learned evidentiary relevance condition; generating, by the ML model, one or more machine-formatted corrective actions to resolve the error condition in the electronic claim, each corrective action comprising structured data conforming to a claim submission standard and linked to the identified documentary evidence; outputting (i) the one or more machine-formatted corrective actions, and (ii) retrievable excerpts of the identified documentary evidence; receiving input indicative of an acceptance of at least one of the machine-formatted corrective actions; and in response to the input, automatically applying the at least one of the machine-formatted corrective actions to the electronic claim, to generate a corrected claim.

In an example, the method further comprises: validating the corrected claim; and in response to validating the corrected claim, filing the corrected claim, to cause the claim to be submitted to an insurance carrier for reimbursement. In an example, the plurality of heterogeneous electronic medical records comprises one or more of (i) a supply chain record including information associated with medical devices used for a medical procedure for the patient encounter, (ii) a clinical note documenting the patient encounter, (iii) a surgical perioperative record, a surgical preoperative record, and/or a surgical postoperative record for the patient encounter, and (iv) a charge posting record including information associated with one or more charges, codes, and/or the claim associated with the patient encounter. In an example, the documentary evidence within the spans supports a corresponding corrective action to resolve the error condition. In an example, the claim is associated with a surgical procedure during the patient encounter; the error condition is associated with a medical implant, which was used during the surgical procedure, missing in the claim; retrieving the plurality of heterogeneous electronic medical records comprises retrieving one or more of (i) a perioperative patient record for the surgical procedure, (ii) a clinical record documenting the surgical procedure, (iii) supply record documenting a supply of the medical implant for the surgical procedure, and (iv) an invoice for the medical implant; identifying the documentary evidence within the spans comprises identifying at least one of the plurality of retrieved heterogeneous electronic medical records that implicitly or explicitly supports use of the medical implant for the surgical procedure; and the corrective actions include an action to add an indication of usage of the medical implant to the claim.

In an example, the error condition indicates that the claim is missing an indication of a location where the patient encounter occurred; retrieving the plurality of heterogeneous electronic medical records comprises retrieving one or more of medical records including the indication of the location where the patient encounter occurred; identifying the documentary evidence within the spans comprises identifying at least one of the plurality of retrieved heterogeneous electronic medical records that includes the indication of the location where the patient encounter occurred; and the corrective actions include adding the indication of the location where the patient encounter occurred to the claim. In an example, the ML model comprises a generative artificial intelligence (AI) model.

In various embodiments, a non-transitory computer-readable medium includes instructions that when executed by one or more processors, cause a system including the one or more processors to perform a set of operations including: receiving, by a computing system, (i) an electronic claim associated with a patient encounter, (ii) a flag indicative of the claim being erroneous, and (iii) a corresponding error report identifying an error condition within the claim; retrieving, by the computing system, a plurality of heterogeneous electronic medical records associated with the patient encounter, the plurality including one or more of structured billing codes, structured data, semi-structured data, and free-text clinical notes; transforming, by a feature extraction engine, the plurality of heterogeneous electronic medical records into a unified machine-readable representation comprising semantic embeddings derived from one or more of the structured data, the semi-structured data, and the free-text clinical notes; processing, by a trained machine learning (ML) model, the unified representation and the error condition to: generate a mapping between the error condition and one or more spans within the unified representation, the mapping determined using (i) learned associations between claim error types and record modalities and/or (ii) the semantic embeddings, and identify documentary evidence within the one or more spans that satisfies a model-learned evidentiary relevance condition; generating, by the ML model, one or more machine-formatted corrective actions to resolve the error condition in the electronic claim, each corrective action comprising structured data conforming to a claim submission standard and linked to the identified documentary evidence; outputting (i) the one or more machine-formatted corrective actions, and (ii) retrievable excerpts of the identified documentary evidence; receiving input indicative of an acceptance of at least one of the machine-formatted corrective actions; and in response to the input, automatically applying the at least one of the machine-formatted corrective actions to the electronic claim, to generate a corrected claim.

In an example, wherein the set of operations include: validating the corrected claim; and in response to validating the corrected claim, filing the corrected claim, to cause the claim to be submitted to an insurance carrier for reimbursement. In an example, the plurality of heterogeneous electronic medical records comprises one or more of (i) a supply chain record including information associated with medical devices used for a medical procedure for the patient encounter, (ii) a clinical note documenting the patient encounter, (iii) a surgical perioperative record, a surgical preoperative record, and/or a surgical postoperative record for the patient encounter, and (iv) a charge posting record including information associated with one or more charges, codes, and/or the claim associated with the patient encounter. In an example, the documentary evidence within the spans supports a corresponding corrective action to resolve the error condition.

In an example, the claim is associated with a surgical procedure during the patient encounter; the error condition is associated with a medical implant, which was used during the surgical procedure, missing in the claim; retrieving the plurality of heterogeneous electronic medical records comprises retrieving one or more of (i) a perioperative patient record for the surgical procedure, (ii) a clinical record documenting the surgical procedure, (iii) supply record documenting a supply of the medical implant for the surgical procedure, and (iv) an invoice for the medical implant; identifying the documentary evidence within the spans comprises identifying at least one of the plurality of retrieved heterogeneous electronic medical records that implicitly or explicitly supports use of the medical implant for the surgical procedure; and the corrective actions include an action to add an indication of usage of the medical implant to the claim. In an example, the error condition indicates that the claim is missing an indication of a location where the patient encounter occurred; retrieving the plurality of heterogeneous electronic medical records comprises retrieving one or more of medical records including the indication of the location where the patient encounter occurred; identifying the documentary evidence within the spans comprises identifying at least one of the plurality of retrieved heterogeneous electronic medical records that includes the indication of the location where the patient encounter occurred; and the corrective actions include adding the indication of the location where the patient encounter occurred to the claim. In an example, the ML model comprises a generative artificial intelligence (AI) model.

In various embodiments, a system comprises: one or more processors; and one or more non-transitory computer-readable media storing instructions, which, when executed by the system, cause the system to perform a set of operations including: receiving, by a computing system, (i) an electronic claim associated with a patient encounter, (ii) a flag indicative of the claim being erroneous, and (iii) a corresponding error report identifying an error condition within the claim; retrieving, by the computing system, a plurality of heterogeneous electronic medical records associated with the patient encounter, the plurality including one or more of structured billing codes, structured data, semi-structured data, and free-text clinical notes; transforming, by a feature extraction engine, the plurality of heterogeneous electronic medical records into a unified machine-readable representation comprising semantic embeddings derived from one or more of the structured data, the semi-structured data, and the free-text clinical notes; processing, by a trained machine learning (ML) model, the unified representation and the error condition to: generate a mapping between the error condition and one or more spans within the unified representation, the mapping determined using (i) learned associations between claim error types and record modalities and/or (ii) the semantic embeddings, and identify documentary evidence within the one or more spans that satisfies a model-learned evidentiary relevance condition; generating, by the ML model, one or more machine-formatted corrective actions to resolve the error condition in the electronic claim, each corrective action comprising structured data conforming to a claim submission standard and linked to the identified documentary evidence; outputting (i) the one or more machine-formatted corrective actions, and (ii) retrievable excerpts of the identified documentary evidence; receiving input indicative of an acceptance of at least one of the machine-formatted corrective actions; and in response to the input, automatically applying the at least one of the machine-formatted corrective actions to the electronic claim, to generate a corrected claim.

In an example, the set of operations include: validating the corrected claim; and in response to validating the corrected claim, filing the corrected claim, to cause the claim to be submitted to an insurance carrier for reimbursement. In an example, the plurality of heterogeneous electronic medical records comprises one or more of (i) a supply chain record including information associated with medical devices used for a medical procedure for the patient encounter, (ii) a clinical note documenting the patient encounter, (iii) a surgical perioperative record, a surgical preoperative record, and/or a surgical postoperative record for the patient encounter, and (iv) a charge posting record including information associated with one or more charges, codes, and/or the claim associated with the patient encounter.

In an example, the documentary evidence within the spans supports a corresponding corrective action to resolve the error condition. In an example, the claim is associated with a surgical procedure during the patient encounter; the error condition is associated with a medical implant, which was used during the surgical procedure, missing in the claim; retrieving the plurality of heterogeneous electronic medical records comprises retrieving one or more of (i) a perioperative patient record for the surgical procedure, (ii) a clinical record documenting the surgical procedure, (iii) supply record documenting a supply of the medical implant for the surgical procedure, and (iv) an invoice for the medical implant; identifying the documentary evidence within the spans comprises identifying at least one of the plurality of retrieved heterogeneous electronic medical records that implicitly or explicitly supports use of the medical implant for the surgical procedure; and the corrective actions include an action to add an indication of usage of the medical implant to the claim. In an example, the error condition indicates that the claim is missing an indication of a location where the patient encounter occurred; retrieving the plurality of heterogeneous electronic medical records comprises retrieving one or more of medical records including the indication of the location where the patient encounter occurred; identifying the documentary evidence within the spans comprises identifying at least one of the plurality of retrieved heterogeneous electronic medical records that includes the indication of the location where the patient encounter occurred; and the corrective actions include adding the indication of the location where the patient encounter occurred to the claim.

In an example, a method comprises receiving (i) a claim, (ii) a flag indicative of the claim being erroneous, and (iii) a corresponding error report, wherein the claim is indicative of charges for a patient encounter, and wherein the error report includes a reason behind the claim being flagged as erroneous; retrieving a plurality of medical records associated with the patient encounter; analyzing, using a machine learning (ML) model, the plurality of medical records, to identify one or more medical records of the plurality of medical records, wherein the identified one or more medical records have documentary evidence that is usable to resolve an error within the claim; causing display of (i) a suggested list of automated actions for resolving the error within the claim, and (ii) at least a section of the one or more medical records indicative of the documentary evidence supporting one or more of the automated actions; receiving an input indicative of an acceptance of the automated actions; and undertaking the automated actions, to resolve the error within the claim and generate a corrected claim. In an example, the method further comprises validating the corrected claim, to ensure that the corrected claim is correct; and filing the claim, to cause the claim to be submitted to an insurance carrier for reimbursement.

In an example, retrieving the plurality of medical records associated with the patient encounter comprises retrieving one or more of (i) a supply chain record including information associated with medical devices used for a medical procedure for the patient encounter, (ii) a clinical note documenting the patient encounter, (iii) a surgical perioperative record, a surgical preoperative record, and/or a surgical postoperative record for the patient encounter, and (iv) a charge posting record including information associated with one or more charges, codes, and/or the claim associated with the patient encounter. In an example, analyzing the plurality of medical records comprises extracting a portion of at least one medical record of the plurality of medical records, wherein the extracted portion of the at least one medical record includes documentary evidence that is usable to resolve the error within the claim. In an example, the claim is associated with a surgical procedure during the patient encounter; the error report indicates that the claim is missing a medical implant used during the surgical procedure; retrieving the plurality of medical records comprises retrieving one or more of (i) a perioperative patient record for the surgical procedure, (ii) a clinical record documenting the surgery procedure, (iii) supply record documenting a supply of the medical implant for the surgery, and (iv) an invoice for the implant medical; analyzing the plurality of medical records comprises identifying that one or more of the plurality of retrieved medical records implicitly or explicitly supports use of the implant for the surgical procedure; and the suggested list of automated actions includes adding the medical implant to the claim.

In an example, the error report indicates that the claim is missing an indication of a location where the patient encounter occurred; retrieving the plurality of medical records comprises retrieving one or more of medical records including the indication of the location where the patient encounter occurred; analyzing the plurality of medical records comprises identifying that one or more of the plurality of retrieved medical records includes the indication of the location where the patient encounter occurred; and the suggested list of automated actions including adding the indication of the location where the patient encounter occurred. In an example, the one or more ML models comprise a generative artificial intelligence (AI) model.

In an example, a non-transitory computer-readable medium includes instructions that when executed by one or more processors, cause a system including the one or more processors to perform operations including receiving (i) a claim, (ii) a flag indicative of the claim being erroneous, and (iii) a corresponding error report, wherein the claim is indicative of charges for a patient encounter, and wherein the error report includes a reason behind the claim being flagged as being erroneous; retrieving a plurality of medical records associated with the patient encounter; analyzing, using one or more machine learning (ML) models, the plurality of medical records, to identify one or more medical records of the plurality of medical records, wherein the identified one or more medical records have documentary evidence that is usable to resolve an error within the claim; causing display of (i) a suggested list of automated actions for resolving the error within the claim, and (ii) at least a section of the one or more medical records indicative of the documentary evidence supporting one or more of the automated actions; receiving an input indicative of an acceptance of the automated actions; and undertaking the automated actions, to resolve the error within the claim and generate a corrected claim.

In an example, a non-transitory computer-readable medium includes instructions that when executed by one or more processors, cause a system including the one or more processors to perform part or all of one or more methods disclosed herein.

In an example, a system comprises: one or more processors; and one or more non-transitory computer-readable media storing instructions, which, when executed by the system, cause the system to perform part or all of one or more methods disclosed herein.

In some embodiments, a system is provided that includes one or more data processors and a non-transitory computer-readable storage medium containing instructions which, when executed on the one or more data processors, cause the one or more data processors to perform part or all of one or more methods disclosed herein.

In other embodiments, a computer-program product is provided that is tangibly embodied in a non-transitory machine-readable storage medium and that includes instructions configured to cause one or more data processors to perform part or all of one or more methods disclosed herein.

Cloud services, microservices, or other machine-hosted services may be offered that perform part or all of one or more methods disclosed herein. The machine-hosted services may be provided by a single machine, by a cluster of machines, or otherwise distributed across machines. The one or more machines may be configured to send and receive data, which may include instructions for performing the methods or results of performing the methods, via an application programming interface (API) or any other communication protocol.

In various embodiments, part or all of one or more methods disclosed herein may be performed by stored instructions such as a software application, computer program, or other software package installed in memory or other storage of a computing platform, such as an operating system, which provides access to physical or virtual computing resources. The operating system may provide access to physical or virtual resources of a mobile computing device, a laptop computing device, a desktop computing device, a server computing device, a container in a virtual machine on a computing device, or any other computing environment configured to execute stored instructions.

As used herein, the terms “first,” “second,” “third,” “fourth,” etc. are used as naming conventions to refer to separate items in a set of items. These naming conventions do not imply ordering unless such ordering is explicitly noted using language specific to ordering, such as “before” or “after,” or unless such ordering is required to attain the expressly recited functionality, such as generating an item and later accessing the generated item.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 illustrates a medical claim generation process flow diagram, in accordance with some examples.

FIG. 2 illustrates a table depicting a plurality of example claim edit errors, and resolution operations performed by an autonomous claim edit system, in accordance with some examples.

FIG. 3 illustrates a medical coding and billing system that includes an autonomous claim edit service for autonomous editing of medical claims, in accordance with some examples.

FIGS. 4A, 4B, 4C, 4D, 4E, 4F, 4G, 4H, 4I, 4J, and 4K illustrate a corresponding plurality of user interfaces (UIs) displaying information associated with autonomous editing of medical claims, in accordance with some examples.

FIG. 5A illustrates a system including an autonomous claim edit service, in accordance with some examples.

FIG. 5B illustrates an operation of a claim error processing service, in accordance with some examples.

FIG. 6 illustrates a method for autonomous editing of medical claims, in accordance with some examples.

FIG. 7 depicts a simplified diagram of a distributed system for implementing certain aspects, in accordance with some examples.

FIG. 8 is a simplified block diagram of one or more components of a system environment by which services provided by one or more components of an embodiment system may be offered as cloud services, in accordance with certain aspects, in accordance with some examples.

FIG. 9 illustrates an example computer system that may be used to implement certain aspects, in accordance with some examples.

### DETAILED DESCRIPTION

Medical coding and billing are error-prone tasks. For example, errors may occur at any stage in the medical coding and billing process. There may be errors within the charges and medical documents associated with the patient encounter, errors associated with the assigned codes, errors occurring during generation of the claims, etc. Such errors are eventually reflected within the claims, which may delay the reimbursement process from the medical insurance carriers.

Accordingly, described herein are techniques for autonomous editing of error resolution of medical claims. For example, an autonomous claim edit service uses rule-based algorithms and/or machine learning (ML) models to autonomously resolve anomalies and errors within medical claims. In an example, using such techniques, claim edits can be streamlined and performed by the autonomous claim edit system. In an example, the autonomous claim edit system proposes corrective actions to cure issues with erroneous claims, and also provides retrievable excerpts of documentary evidence supporting the proposed corrective actions. In an example, the billers may review corrective actions proposed by the autonomous claim edit service, and choose to accept or decline the proposed resolution. Once accepted, the autonomous system executes the resolution steps for editing and correcting the claims, and/or underlying charges and/or assigned codes. Accuracy of such claim edit process may be achieved by the autonomous claim edit system. Such as autonomous claim edit system is scalable, and may autonomously process a large amount of medical claims, along with retrieving and providing documentary evidence supporting each such corrective actions.

In further detail, in a healthcare billing pipeline, a patient is registered for a patient visit, also referred to as a patient encounter. The patient visit can be to meet with a healthcare provider (such as a physician, a nurse, a medical technician, etc.), for an outpatient visit, for an inpatient visit (e.g., admitted in a hospital), for a procedure visit (such as for a CT scan), for a laboratory testing, and/or for any other types of encounter between a patient and a medical care professional. After or during the patient encounter, charges are generated based on clinical activity and documents indicative of the patient visit, where the charges include medical record documentation, such as transcription of clinical or surgical notes or after-visit summary captured by medical care professionals, laboratory and radiologic results, supply list and/or invoice of medical equipment and medical implants used for a medical procedure, etc.

After the charges are generated, medical coding for the patient encounter is performed. For example, medical coders and/or an autonomous coding system assign medical codes associated with the patient encounter. For example, one or more medical codes are assigned to the patient encounter, based on the charges and other documents associated with the patient encounter. Errors detected in the patient registration process, charge generation process, and/or code assignment process may be appropriately resolved.

Subsequently, for each patient encounter, one or more claims are generated from the assigned codes. The claims may be generated by medical coders, billers, and/or autonomously by one or more rule-based algorithms, one or more machine learning (ML) models employing artificial intelligence (AI), and/or the like. Subsequently, the claims are validated by a claim validation service, to ensure that these are correct. For example, the validation of the claims may be performed manually, may be performed by one or more rule-based algorithms, and/or one or more ML models. During the validation, possible anomalies and errors in one or more medical claims are flagged.

In an example, the autonomous claim edit service receives claims that are flagged to be erroneous by the claim validation service, along with an error report including reasons behind flagging each claim as being erroneous (such as identifies an error condition within the claim).

For each erroneous claim, the autonomous claim edit service retrieves a plurality of medical records that are managed by a records management system, in an example. For example, when processing an erroneous claim, the autonomous claim edit service retrieves medical records associated with a patient encounter for the claim in question.

Examples of such medical records may include supply chain records, e.g., including supply chain information, invoices, etc. associated with equipment, medical devices, etc. used for medical procedures (such as surgeries), for which the patient and/or the insurance carrier are to be billed. Examples of such medical records may further include clinical notes and documents entered by healthcare providers (such as physicians, surgeons, nurses, medical technicians, etc.) prior to, during, and/or subsequent to a patient encounter. Examples of such medical records may also include surgical records entered by healthcare providers (such as physicians, surgeons, nurses, medical technician, etc.) prior to, during, and/or subsequent to a patient encounter that involves a surgery or a medical procedure, such as perioperative patient record (periop records), preoperative records, postoperative records, etc. Examples of such medical records may also include financial data entered after a patient encounter, such as charges, codes, claims, etc.

Thus, the retrieved records are heterogeneous electronic medical records associated with the patient encounter. In an example, the records includes one or more of structured billing codes, structured data, semi-structured data, and free-text clinical notes, as described below.

Subsequently, the autonomous claim edit service calls a claim error processing service to perform review of the retrieved records, and suggest automated corrective actions based on such review. In an example, the claim error processing service comprises a feature extraction engine, which processes the records, and transforms the heterogeneous medical records into a unified machine-readable representation comprising semantic embeddings derived from one or more of the structured data, the semi-structured data, and un-structured free-text clinical notes. For example, the feature extraction engine acts as a unifying layer on the structured data, semi-structured data, and free-text clinical notes, and/or normalizes and represents the heterogeneous records in a format that a machine learning (ML) model can consume. In an example, the ML model analyzes the records and/or the corresponding semantic embeddings in view of the error condition. For example, the ML model generates a mapping between the error condition and one or more spans within the unified representation of the semantic embeddings. In an example, the mapping may be determined by the ML model using (i) learned associations between claim error types and record modalities and/or (ii) the semantic embeddings.

For example, the records have a plurality of record modalities. For example, the records comprises one or more of supply chain records, clinical notes, periop records, preoperative records, postoperative records, and/or any other relevant records associated with the patient encounter. The ML model may be trained to look at a specific record modality, e.g., based on a type of the error condition. For example, if the error condition is associated with a missing implant charges, then the ML model may parse and analyze supply chain records and periop records, to find documentary evidence implicitly and/or explicitly supporting use of the implant during the patient encounter. In an example, the ML model generates the mapping between the error condition and one or more spans within the unified representation, and based on such a mapping, identifies documentary evidence within the one or more spans that satisfies a model-learned evidentiary relevance condition. In the above example where the error condition is associated with the missing implant charges, the ML model identifies documentary evidence within the supply chain records and/or periop records (or within another record modality), which implicitly and/or explicitly supports use of the implant during the patient encounter.

Based on historical data and current information, the ML model may suggest corrective automated actions for potential resolution of the erroneous claim. For example, the ML model generates one or more machine-formatted corrective actions to resolve the error condition in the claim, wherein each corrective action comprises structured data conforming to a claim submission standard and linked to the identified documentary evidence. Thus, a corrective action, if implemented, would conform the corrected claim to the claim submission standard. For example, the ML model may tailor the suggested corrective actions based on a nature of the error. For example, for the above-described example where implant charges are missing, the ML model identifies a plurality of records that implicitly and/or explicitly indicate usage of the implant during the surgery, so that suggested automated actions may include generating implant charges. The ML model identifies documentary evidence within the retrieved records, to support the suggested automated actions.

In an example, the ML model outputs (i) the one or more machine-formatted corrective actions, and (ii) retrievable excerpts of the identified documentary evidence. After the ML model outputs the corrective actions and the user (such as a coder or a biller) agrees to such automated actions, the claim is corrected. For example, the autonomous claim edit service receives user input indicative of an acceptance of at least one of the machine-formatted corrective actions, and in response to the input, automatically applies the at least one of the machine-formatted corrective actions to the electronic claim, to generate the corrected claim.

The corrected claim is transmitted to a claim validation service, for revalidation of the corrected claim. If the claim validation service successfully validates the claims, the corrected claim is now ready for submission to the insurance carrier. After the corrected claim has been validated, the corrected claim is transmitted to a charge posting system. The corrected claim is then filed, e.g., submitted to the medical insurance carrier for reimbursement.

One of the technical challenges addressed by some embodiments of the disclosure relates to identifying corrective actions for an erroneous claims, and also identifying documentary evidence supporting such corrective actions. Because each claim is associated with a large number of records for the corresponding patient encounter, it may be challenging for a traditional system to pinpoint a specific record (or a specific portion of a record) supporting corrective actions to correct an erroneous claim. Even with access to computational tools, a human attempting to manually parse large volume of records, to map (i) an error condition and (ii) a section of a record that supports correcting the error correction would face severe limitations in speed, consistency, and scalability—especially when dealing with diverse types of error conditions and high-volume claim streams.

A technical solution provided by some embodiments includes techniques that leverage on retrieval of heterogenous relevant records including structured, semi-structured, and unstructured data, transformation of the heterogeneous medical records into a unified machine-readable representation comprising semantic embeddings, and mapping between the error condition and one or more spans within the unified representation of the semantic embeddings across the record modalities. Such mapping of the error condition and one or more spans within one or more of a plurality of record modalities not only facilitate generation of proposed corrective actions to correct an erroneous claim, but also enables identification of documentary evidence within the one or more spans that satisfies a model-learned evidentiary relevance condition and supports the corrective actions. The techniques are easily scalable and supports any possible error conditions associated with erroneous claims, as long as the error conditions are curable based on available reports that are associated with the patient encounter. This approach provides a technical advantage of dynamically generating proposed corrective actions for erroneous claims, where each such proposed corrective action is supported by documentary evidence.

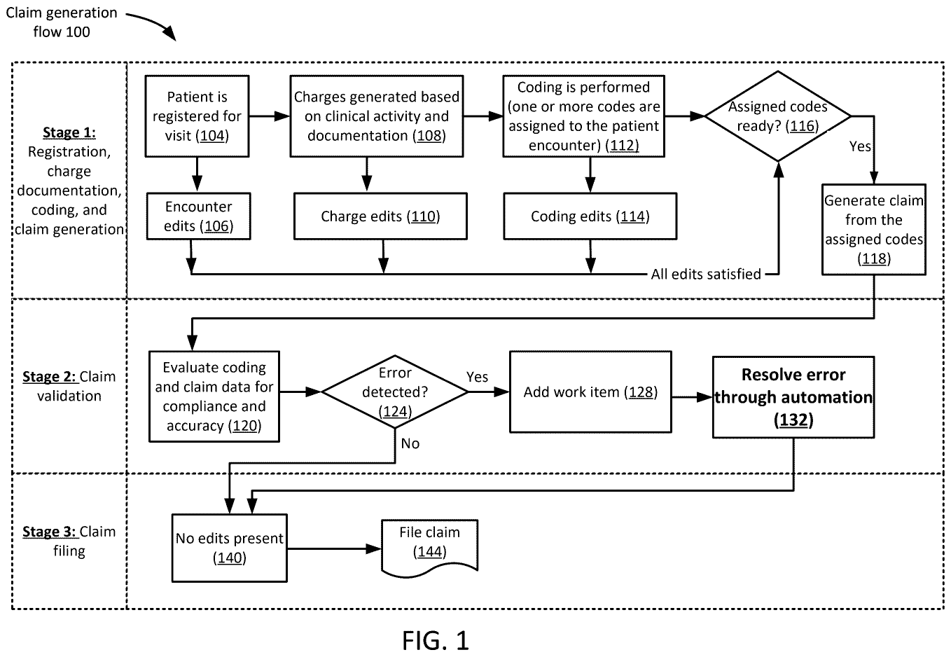

FIG. 1 illustrates a medical claim generation process flow diagram 100, in accordance with some examples. As illustrated in FIG. 1, in an example, generation and filing of a medical claim can broadly be divided in three stages, such as (i) a stage 1 in which patient registration, charge documentation, coding, and claim generation are performed, (ii) a stage 2 in which the claim data are validated, and (iii) a stage 3 in which the claim is filed. FIG. 1 outlines these three stages.

For example, referring to the stage 1 in FIG. 1, at 104, a patient is registered for a patient visit, also referred to as a patient encounter. The patient visit can be to meet with a healthcare provider (such as a physician, a nurse, a medical technician, etc.), for an outpatient visit, for an inpatient visit (e.g., admitted in a hospital), for a procedure visit (such as for a CT scan), for a laboratory testing, and/or for any other types of encounter between a patient and a medical care professional.

After or during the patient encounter, charges are generated based on clinical activity and documents indicative of the patient visit, labelled as 108 in FIG. 1. The charges include medical record documentation, such as transcription of clinical or surgical notes or after-visit summary captured by medical care professionals, laboratory and radiologic results, supply list and/or invoice of medical equipment and medical implants used for a medical procedure, etc. The charges may be generated by healthcare professionals (also referred to as medical professionals), such as physicians, surgeons, anesthesiologists, nurses, medical technicians, diagnostic and laboratory equipment operators, etc. The charges, for example, are indicative of an amount of time a doctor spent with the patient, a diagnosis provided by the doctor, a type of procedure conducted on the patient, a type of laboratory test conducted on the patient, a list of medical equipment used during a medical procedure, and/or any other relevant information that may be relied upon for billing the patient and/or an insurance carrier insuring the patient. The charges, among other things, summarize and document the patient visit. The charges may be generated from electronic health record (EHR) documenting the patient encounter, and/or may comprise the EHR itself.

After the charges are generated, at 112, medical coding for the patient encounter is performed. For example, medical coders and/or an autonomous coding system assign the medical codes. For example, one or more medical codes are assigned to the patient encounter, based on the charges and other documents associated with the patient encounter.

In an example, at 116, a determination is made as to whether the codes are now complete and ready for claim generation. For example, there may be inadvertent errors in the processes 104, 108, and/or 112, which has to be edited prior to claim generation. For example, any intended edits in documenting the patient registration process may be performed at 106 (labelled as patient encounter edits in FIG. 1). Similarly, for example, any intended edits in generating the charges may be performed at 110 (labelled as charge edits in FIG. 1). Finally, for example, any intended edits in the assigned codes may be performed at 114 (labelled as coding edits in FIG. 1).

Once the edits, if needed, are performed, and the codes are assigned, the assigned codes are now ready for generation of the claims. For example, at 118, for each patient encounter, one or more claims are generated from the assigned codes. The claims may be generated by medical coders, billers, and/or autonomously by one or more rule-based algorithms, one or more ML models employing artificial intelligence (AI), and/or the like.

Subsequently, the process flow 100 proceeds from 118 of stage 1 to stage 2, in which the assigned codes and claim information are validated, to ensure that these are correct. For example, at 120, an evaluation is made as to whether the coding and/or claim data are accurate and meets compliance requirements. In an example, the validation at 120 may be performed manually, may be performed by one or more rule-based algorithms, one or more ML models employing artificial intelligence (AI), and/or the like. Thus, at 120, possible anomalies and errors with the assigned codes and resultant medical claim are flagged.

At 124, a determination is made as to whether any error is detected to the codes and claims assigned to a specific patient encounter. If “No” at 124, the process flow proceeds to 140 of stage 3. However, if “Yes” at 124, the process flow proceeds to 128, where the detected erroneous claims are added to a work item for resolution. Thus, multiple such erroneous claims corresponding to multiple patient encounters are added as work items.

At 132, the errors are resolved through automation using one or more ML models, as will be described below in further detail. Thus, once possible errors in assigned codes and/or claims are identified at 120, such errors are resolved at 132 using various techniques described herein, in an example.

Once the errors are resolved at 132, the process flow 100 proceeds from 132 to 140. At 140, it is verified that no edits to the claims are to be made. Accordingly, at 144, the claim is filed, e.g., submitted to medical insurance carriers for reimbursement.

Thus, at 132 of the process flow 100, errors and anomalies detected within assigned codes and/or claims are resolved. In an example, using techniques described below, a large percentage of such claim edits can be performed by an autonomous claim edit system, e.g., using AI and ML models. For example, a large number of claim edits, which require charge and registration related edits, can be processed by ML models described below. In an example, the billers may review the proposed resolution and accept or decline the edits proposed by the autonomous system. Once accepted, the autonomous system executes the resolution steps for editing the claims, and/or underlying charges and/or assigned codes, without any substantial or any manual intervention. Accuracy of such claim edit process may be achieved by the autonomous claim edit system.

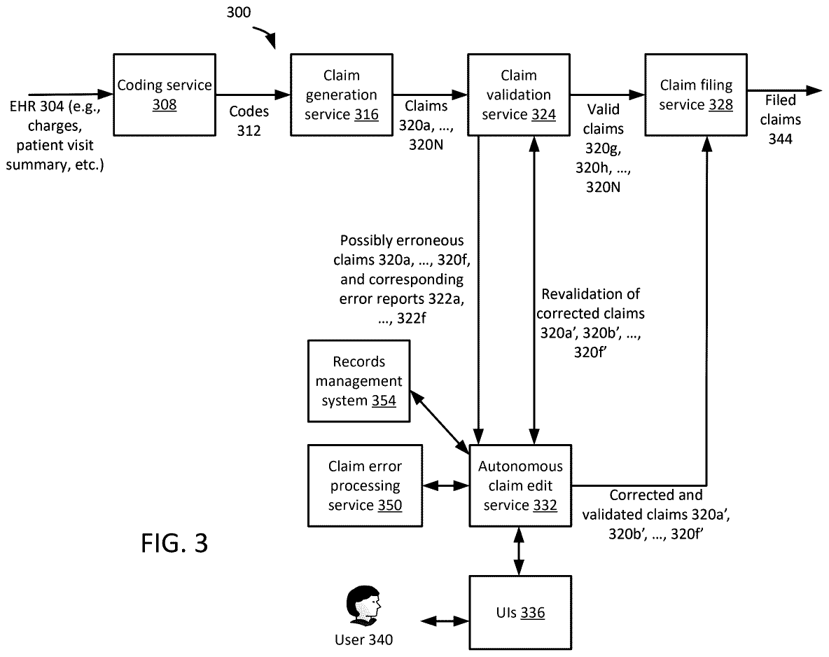

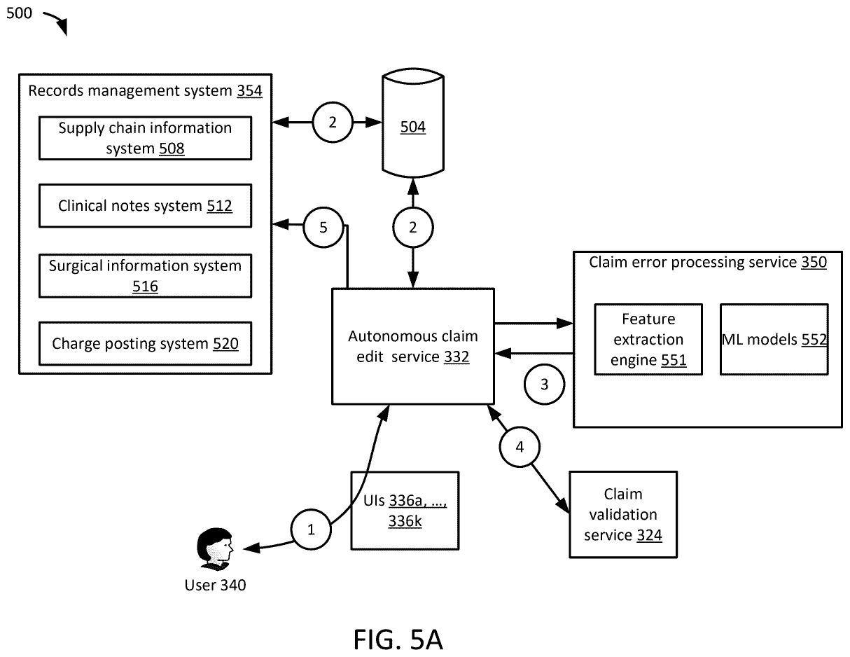

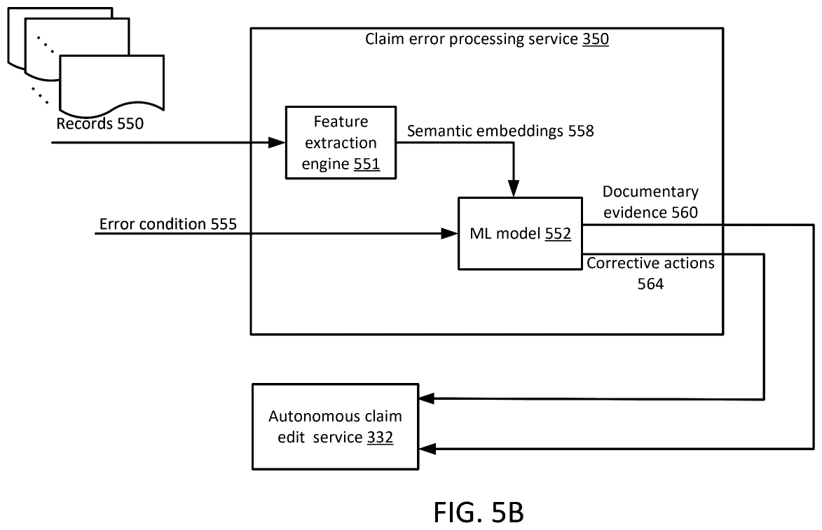

As described below in further detail (see FIGS. 3 and 5A, 5B below), the autonomous claim edit system may use a blended automation approach. For example, the autonomous claim edit system may use rule-based approach, along with ML models, for claim editing. For example, ML models may be used to review patient records and answer contextual questions (e.g. whether patient summary indicates moderate level of Evaluation and management (E/M) services, where a E/M service provided by a physician or other health care professional in which the provider is either evaluating or managing the health of a patient). In an example, rules-based automation may be used to merge and/or split charges, manage assigned codes based on payer rules, and/or the like.

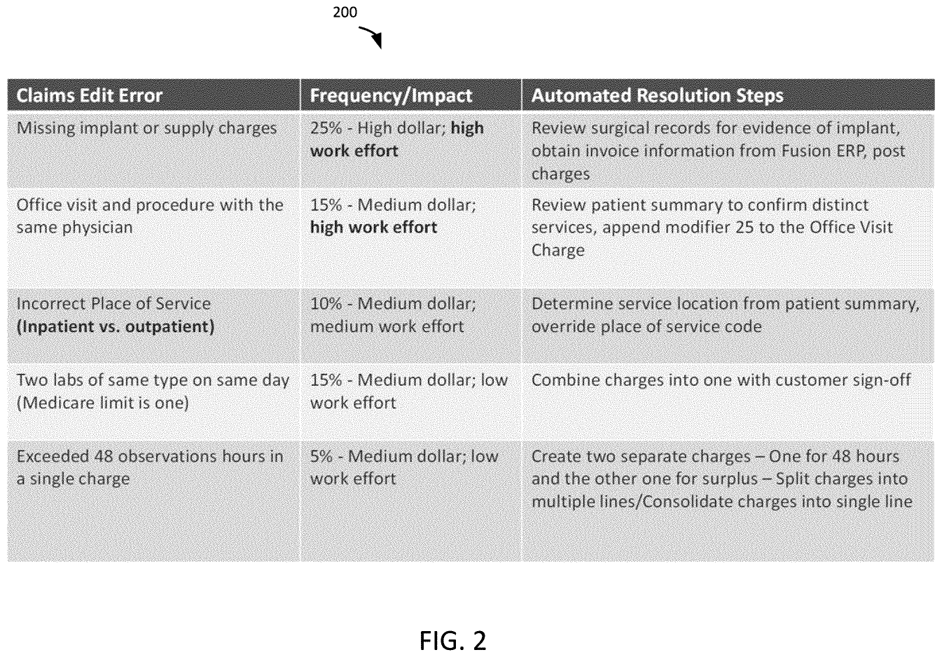

FIG. 2 illustrates a table 200 depicting a plurality of example claim edit errors, and resolution operations performed by the autonomous claim edit system described below with respect to FIGS. 3 and 5A, 5B, in accordance with some examples. FIG. 2 will be self-evident, based on the discussion herein.

FIG. 3 illustrates a medical coding and billing system 300 that includes an autonomous claim edit service 332 for autonomous editing of claims, in accordance with some examples. The autonomous claim edit service 332 is also referred to as a billing exception resolution application, as the service 332 aims to resolve anomalies in claims that are to be billed.

The system 300 includes a coding service 308 that receives electronic health records (EHR) 304. The EHR 304 associated with a patient encounter comprises documents and records generated based on the patient encounter, such as charges, patient visit summary, clinical and surgical notes, supply and invoice reports documents medical equipment used for medical procedures, laboratory results, medical test results, medical procedure results, invoices of material used for medical procedure, and/or other relevant documents used for assigning codes and bills for a patient encounter, for example.

The coding service 308 assigns one or more codes 312 to each patient encounter. The coding service 308 may be operated by one or more medical coders, and/or may be an autonomous coding service executed by one or more ML models.

The system 300 includes a claim generation service 316 that receives, for each patient encounter, one or more assigned codes 312, and generates a corresponding claim 320. For example, a plurality of claims 320a, . . . , 320N are generated corresponding to a plurality of patient encounters. The claim generation service 316 may be operated by one or more medical coders and/or billers, and/or may be an autonomous billing service executed by one or more ML models, in an example.

The claims 320a, . . . , 320N are validated by a claim validation service 324. The claim validation service 324 may be operated by one or more medical coders and/or billers, and/or may be an autonomous validation service executed by one or more ML models. For example, an autonomous validation service assigns, for a claim, a probability of the claim being valid. If this probability is higher than a threshold value, the claim is considered to be valid or correct. On the other hand, if this probability is lower than the threshold value, the claim is considered to be possibly erroneous or invalid. Various variations in the operation of the claim validation service 324 may be possible. In an example, the claim validation service 324 employs statistical and/or probabilistic ML models, generative AI models, and/or LLM models for assigning the probability to individual claims.

In the example of FIG. 3, the claim validation service 324 validates a plurality of claims 320g, . . . , 320N as being valid or correct claims. The claims 320g, . . . , 320N, which are declared as being valid, are transmitted to a claim filing service 328 for filing of the claims, e.g., by submitting the claims (labelled as filed claims 344) to medical insurance carriers for reimbursement. The filing service 328 may be operated by one or more medical coders and/or billers, and/or may be an autonomous filing service executed by one or more AI/ML models.

The claim validation service 324 also flags another plurality of claims 320a, . . . , 320f as being erroneous (or being anomalous). For each of the claims 320a, . . . , 320f flagged as being erroneous, the claim validation service 324 also outputs a corresponding error report 322 that indicates a type of error for the corresponding claim. For example, error report 322a indicates a type of error (or a reason behind the error flag) detected by the claim validation service 324 for the claim 320a. Thus, the claim validation service 324 flags the claims 320a, . . . , 320f as being erroneous, and correspondingly also outputs the error reports 322a, . . . , 322f.

The flagging of the claims 320a, . . . , 320f as being erroneous and the corresponding error reports 322a, . . . , 322f are received by the autonomous claim edit service 332. In an example, the autonomous claim edit service 332 causes to display, on one of a plurality of user interfaces (UIs) 336, the erroneous claims 320a, . . . , 320f, and/or one or more reasons for each of the displayed claims being flagged as erroneous. One or more users 340 (such as medical coders and/or billers) can view and interact with the plurality of UIs 336.

In an example, the autonomous claim edit service 332 interacts with a claim error processing service 350 and a records management system 354, e.g., to resolve the erroneous claims 320a, . . . , 320f reported by the claim validation service 324, as described below in further detail.

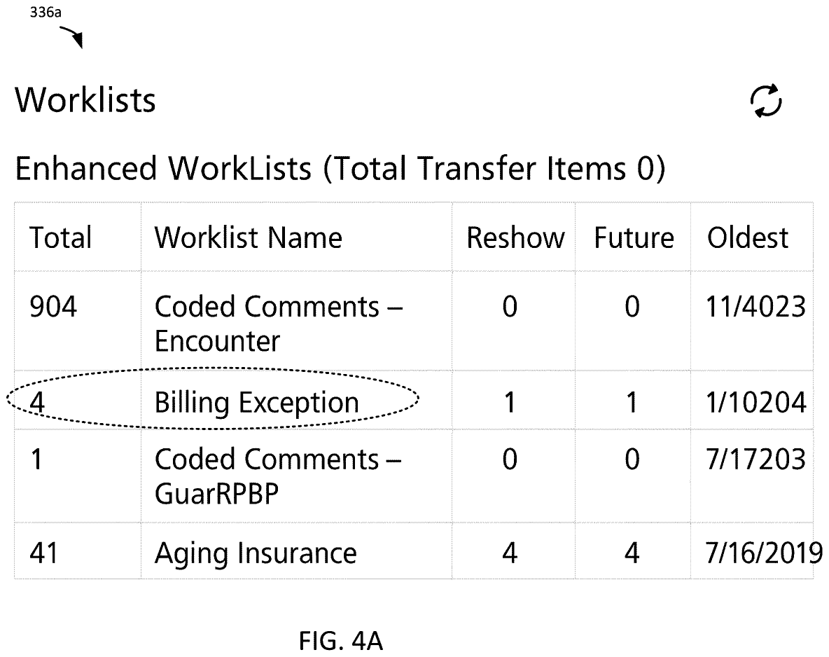

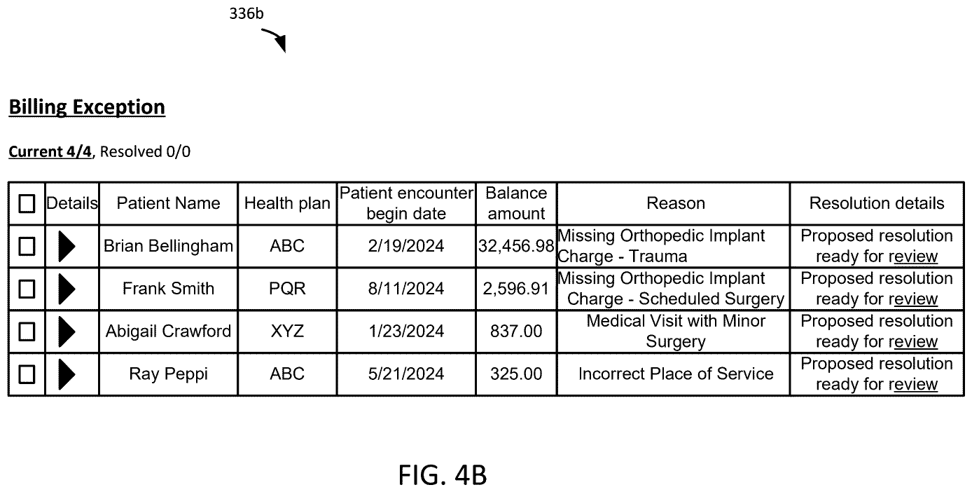

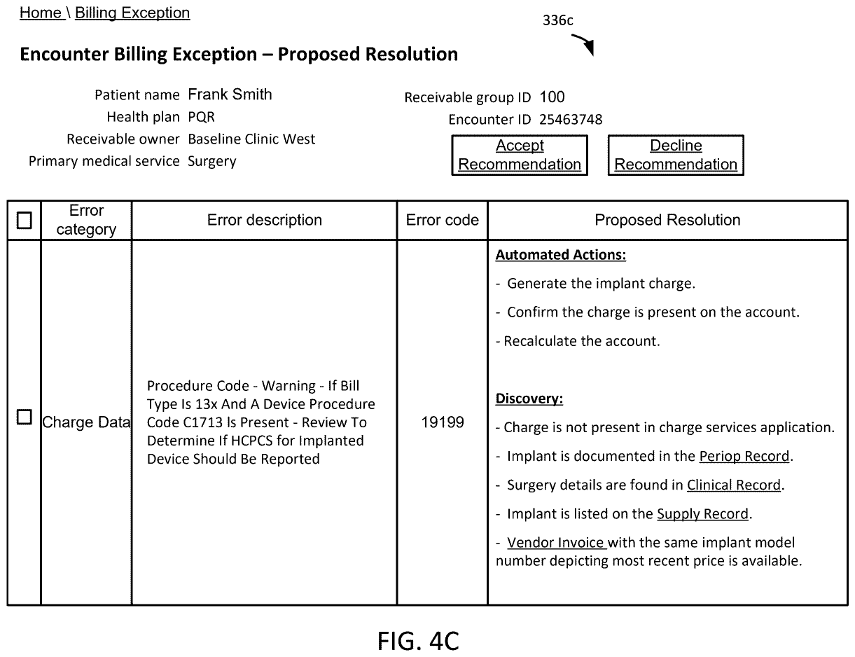

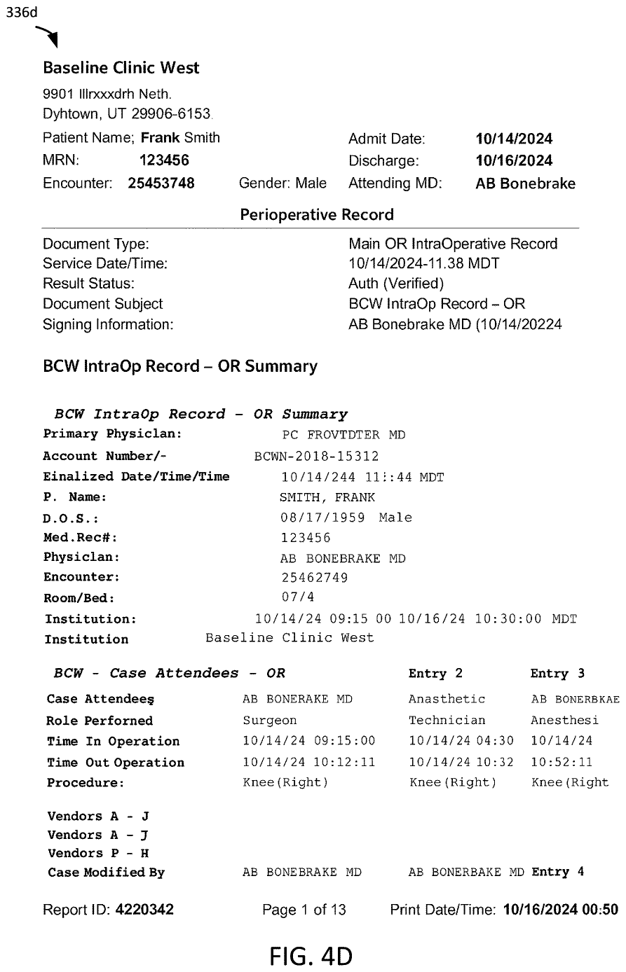

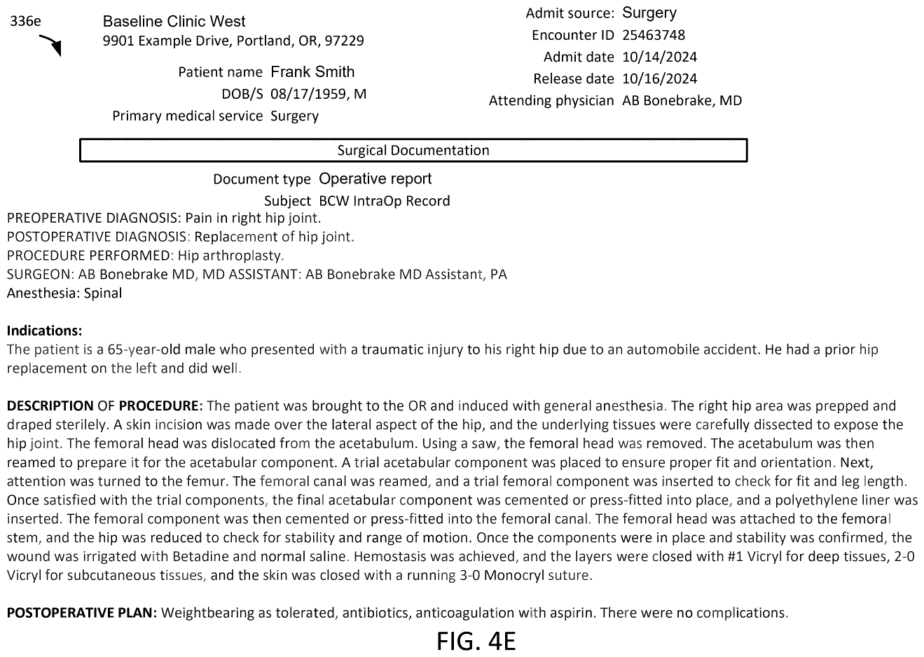

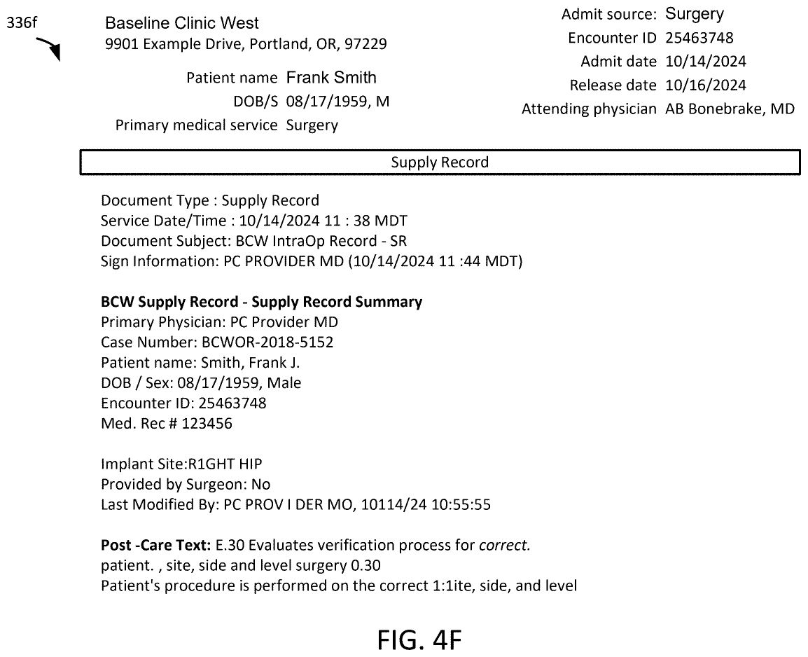

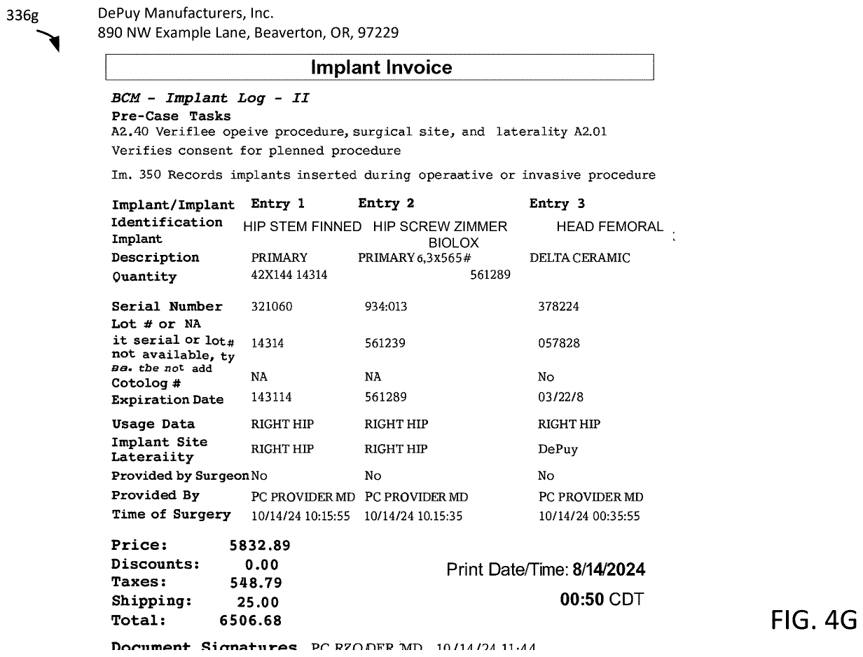

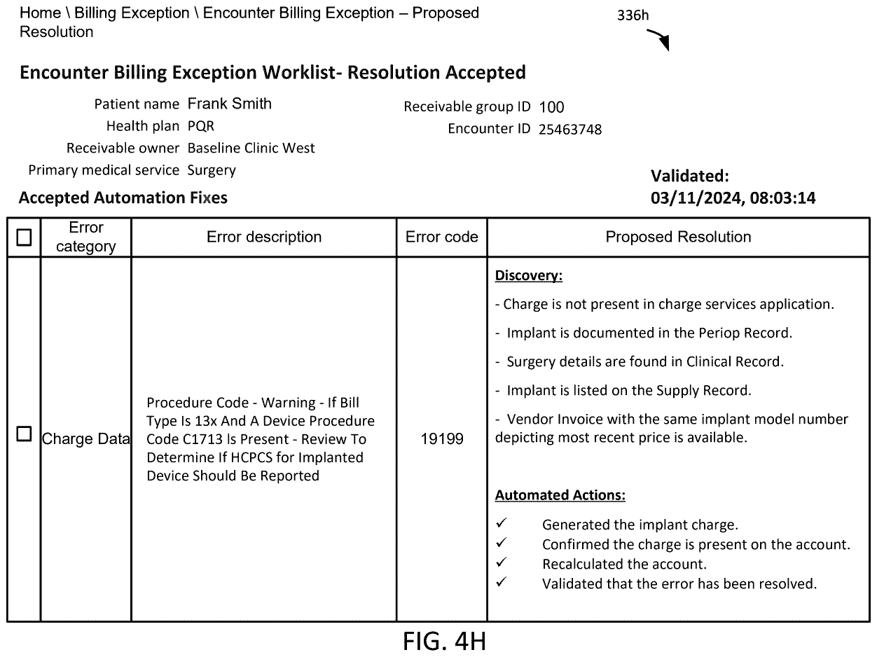

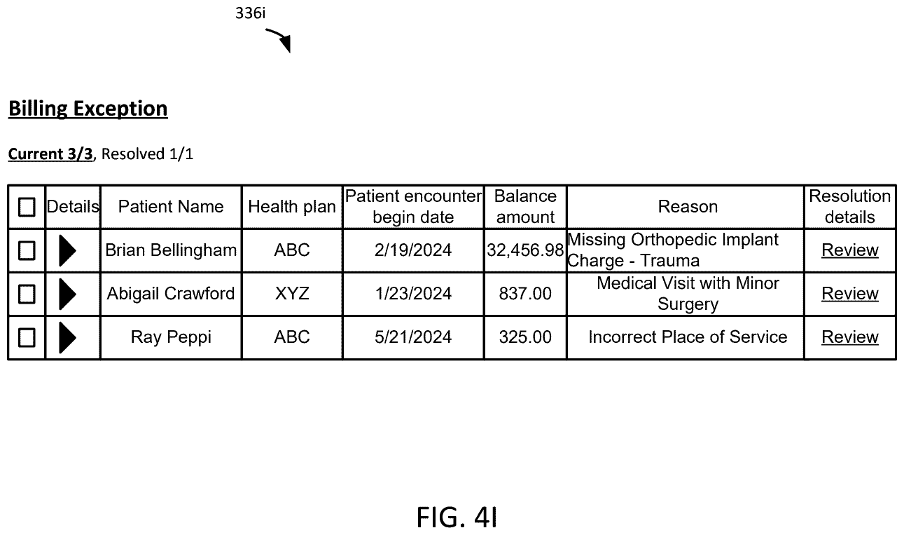

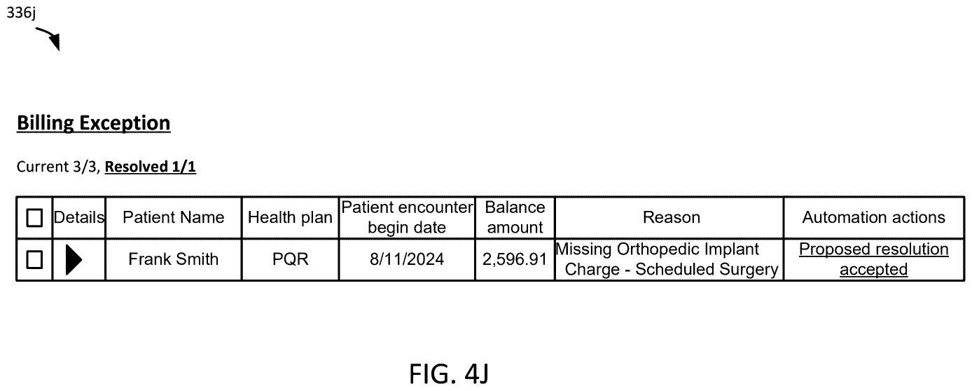

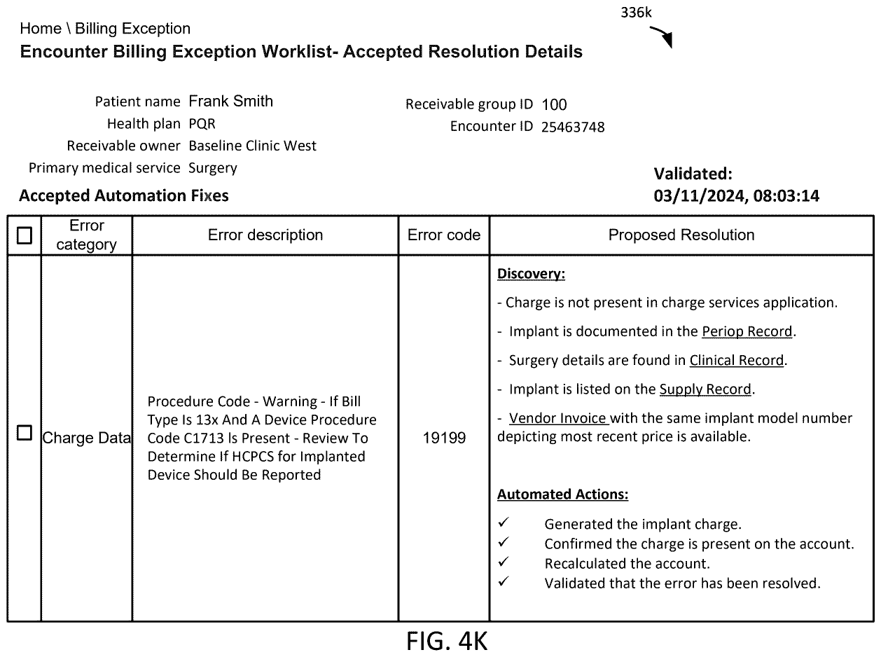

FIGS. 4A, 4B, 4C, 4D, 4E, 4F, 4G, 4H, 4I, 4J, and 4K illustrate a corresponding plurality of UIs displaying information associated with autonomous editing of medical claims, in accordance with some examples. In an example, the autonomous claim edit service 332 causes generation and display of one or more of the UIs of FIGS. 4A-4K.

FIG. 4A illustrates a UI 336a displaying an option to view possible erroneous claims received by the autonomous claim edit service 332. For example, as indicated by the imaginary doted oval shape in FIG. 4A, displayed is an option within the UI 336a to view “Billing Exceptions.” Here billing exceptions implies claims that are flagged as being erroneous, such as claims 320a, . . . , 320f, which may eventually cause billing issues. The example of FIG. 4A also illustrates that four claims have billing exceptions (“Total” is 4 in the first column, second row of the table in the UI 336a).

FIG. 4B illustrates a UI 336a displaying a plurality of billing exceptions. For example, the UI 336b is launched, based on the user 340 selecting the “Billing Exception” option of the UI 336a of FIG. 4A. FIG. 4B illustrates four example claims having billing exceptions. For each such claim, the UI 336a illustrates a patient name (note that the patent name are fictitious for purposes of patient confidentiality), a corresponding insurance carrier name and a corresponding health plan, a start date of the patient encounter, a dollar amount associated with the claim, and a reason the claim is flagged by the claim validation service 324 as being erroneous.

The UI 336b also has, for each claim within the UI 336b, an option to view a resolution proposed by the autonomous claim edit service 332. For example, the last column of the table of the UI 336b is for “Resolution Detail,” and selecting “Review” in this column for any of the claims would display a manner in which the autonomous claim edit service 332 would resolve the anomaly or error in the corresponding claim.

FIG. 4C illustrates a UI 336c displaying a resolution or edit proposed by the autonomous claim edit service 332 for an erroneous claim. For example, the UI 336c is for the patient “Frank Smith,” which is the second listed patient in the table of the UI 336b of FIG. 3B. Selecting the “Review” option in the “Resolution Details” column for this patient within the UI 336b of FIG. 3B causes display of the UI 336c of FIG. 3C.

Within the UI 336c, the user 340 can review the error information and the automated resolution recommendations proposed by the autonomous claim edit service 332. The user 340 is provided with an option to either accept or decline the recommendations.

As illustrated in FIG. 4B, the error associated with the claim for the patient Frank Smith is “Missing Orthopedic Implant Charge - Scheduled Surgery.” Accordingly, the error is associated with missing charges for orthopedic implant in the claim for an orthopedic surgery. The user 340 can select the links presented in the UI 336c of FIG. 4C to access (i) perioperative patient record (Periop record), (ii) clinical record, (iii) supply record (e.g., a list of medical and/or surgical equipment used for the medical procedure, such as the orthopedic implants used for the surgery), and/or (iv) vendor invoice for such equipment, as illustrated in the right bottom corner of the UI 336c.

FIG. 4D illustrates a UI 336d displaying an example perioperative patient record (Periop record). The Periop record is for the patient Frank Smith, and the UI 336d is displayed based on a user selecting the “Implant is documented in the Periop record” link from the lower right side of the UI 336c of FIG. 4C. For example, the autonomous claim edit service 332 detects that the implant is recorded in the Periop record, and the user 340 can view documentary evidence of the implant use from the Periop record in the UI 336d of FIG. 4D. For example, the procedure “Arthroplasty Replacement Total” indicated in the Periop record of the UI 336d of FIG. 4D implies that implant was used in the surgery.

FIG. 4E illustrates a UI 336e displaying an example clinical record. The clinical record is for the patient Frank Smith, and the UI 336e is displayed based on a user selecting the “Surgery details are found in Clinical Record” link from the lower right side of the UI 336c of FIG. 4C. For example, the autonomous claim edit service 332 detects that the clinical record includes the surgery details, and hence, provides a link to the user 340 for viewing the clinical record.

FIG. 4F illustrates a UI 336f displaying an example supply record for an implant. The supply record is for the patient Frank Smith, and the UI 336f is displayed based on a user selecting the “Implant is listed in the Supply Record” link from the lower right side of the UI 336c of FIG. 4C. For example, the autonomous claim edit service 332 detects that the implant is listed in the supply record, and the user 340 can view evidence of the implant use from the supply record in the UI 336f of FIG. 4F.

FIG. 4G illustrates a UI 336g displaying an example invoice for an implant. The invoice is for the implant used on patient Frank Smith, and the UI 336f is displayed based on a user selecting the “Vendor Invoice with the same implant model number depicting most recent price is available” link from the lower right side of the UI 336c of FIG. 4C. For example, the autonomous claim edit service 332 detects that the implant is invoiced in the invoice record, and the user 340 can view documentary evidence of the implant invoice from the invoice record in the UI 336g of FIG. 4G.

Referring again to the UI 336c of FIG. 4C, this UI 336c also lists proposed automated corrective actions that the autonomous claim edit service 332 can undertake. For example, as illustrated in the UI 336b and UI 336c, the error associated with the claim for the patent Frank Smith is associated with missing orthopedic implant charge for a surgery procedure. For example, an error report 322 for this claim, as generated by the claim validation service 324, reports the missing orthopedic implant charge.

The autonomous claim edit service 332 and/or the claim error processing service 350, upon reviewing the error report 322 for this claim, recognize the missing orthopedic implant charge error. In response, the autonomous claim edit service 332 and/or the claim error processing service 350 review a plurality of records from the records management system 354. The records reviewed may be associated with the patient encounter, such as the periop record, clinical record, the supply record, the vendor record, preoperative record, postoperative record, and/or other one or more relevant records associated with the patient encounter. Because the patient has undergone a complex surgical procedure, multiple such records exist for the patient encounter.

The autonomous claim edit service 332 and/or the claim error processing service 350, upon reviewing such records from the records management system 354, identify one or more records that can resolve the error indicated in the error report. In this example, the autonomous claim edit service 332 and/or the claim error processing service 350 identifies the periop record, the clinical record, the supply record, and the invoice (illustrated respectively in FIGS. 4D-4G), each of which implicitly or explicitly indicates use of implant during the procedure. Thus, autonomous claim edit service 332 and/or the claim error processing service 350 identify a plurality of records that can resolve the error associated with the missing implant charge, as the autonomous claim edit service 332 and/or the claim error processing service 350 have now positively identified use of the implant during the procedure.

Accordingly, as illustrated in UI 336c of FIG. 4C, the autonomous claim edit service 332 causes to display one or more automated actions that the autonomous claim edit service 332 can undertake to resolve the error. For example, the automated actions listed in FIG. 4C includes (i) generating implant charges, (ii) confirming that the charge is present on the account, and (iii) recalculate the account. As also described above, the autonomous claim edit service 332 also causes to display documentary evidence supporting the proposed automated actions (see the above-described links on the right lower side of the UI 336, such as links for the periop record, clinical record, the supply record, and the vendor record).

In the UI 336c of FIG. 4C, the autonomous claim edit service 332 also presents an option to accept the recommendation of the automated actions, or to decline the recommendation of the automated actions. Upon the user 340 selecting the option to decline the recommendation of the automated actions, no automated actions are undertaken by the autonomous claim edit service 332, in an example.

On the other hand, upon the user 340 selecting the option to accept the recommendation of the automated actions, a UI 336h of FIG. 4H is displayed. FIG. 4H illustrates a UI 336h displaying automated actions performed by the autonomous claim edit service 332 and/or the claim error processing service 350, to resolve the erroneous claim flagged by the claim validation service 324. For example, in the UI 336h, each of the automated actions (i) generating implant charges, (ii) confirming that the charge is present on the account, and (iii) recalculating the account are indicated by corresponding checkmarks, thereby indicating that the autonomous claim edit service 332 has undertaken these corrective actions.

Assume that the original claim was 320a, which was flagged by the claim validation service 324 as being erroneous. Upon completing the corrective actions by the autonomous claim edit service 332 and/or the claim error processing service 350, assume the corrected claim is 320a′ (also see FIG. 3). The autonomous claim edit service 332 revalidates the corrected claim 320a′ from the claim validation service 324 (symbolically illustrated using a line with arrows on both side between the claim validation service 324 and the autonomous claim edit service 332 in FIG. 3). Assuming that the claim validation service 324 validates the corrected claim 320a′ as being correct, the autonomous claim edit service 332 causes to display on the UI 336h of FIG. 4H the following” “Validated that the error has been resolved,” along with a corresponding checkmark. The error of the claim 320a is now resolved, as a corrected claim 320a′ is now generated.

Referring again to FIG. 3, the autonomous claim edit service 332 transmits a plurality of such corrected and revalidated claims 320a′, 320b′, . . . , 320f′ to the claim filing service 328. The claim filing service 328 in turn submits these corrected and validated claims 320a′, 320b′, . . . , 320f′ to respective medical insurance carriers for reimbursement.

FIG. 4I illustrates a UI 336i indicating a list of unresolved erroneous claims, and any resolved claims are no longer present in the list of unresolved erroneous claims. For example, comparing the UI 336b of FIG. 4B and the UI 336i of FIG. 4I, the erroneous claim for the patient Frank Smith was present in the UI 336b of FIG. 4B. However, this claim is edited and resolved by the autonomous claim edit service 332, as described above with respect to FIGS. 4C-4H. Accordingly, in the UI 336i of FIG. 4I, the claim for this patient Frank Smith is no longer present, in an example.

FIG. 4J illustrates a UI 336j indicating a list of resolved claims. For example, the claim for the patient Frank Smith is displayed in the UI 336j as being resolved.

FIG. 4K illustrates a UI 336k indicating a list of automated actions performed by the autonomous claim edit service 332 to resolve a claim. For example, UI 336k of FIG. 4K can be displayed by selecting “Proposed Resolution Accepted” link of the UI 336j of FIG. 4J. The UI 336k of FIG. 4K displays the list of automated actions performed by the autonomous claim edit service 332 to resolve the claim for the patient Frank Smith, as described above.

FIG. 5A illustrates a system 500 including an autonomous claim edit service 332, in accordance with some examples. FIG. 5B illustrates an operation of the claim error processing service 350 that interacts with the autonomous claim edit service 332, to resolve claims errors, in accordance with some examples. FIGS. 5A and 5B will be described in unison.

As described above with respect to FIG. 3 and as also illustrated in FIG. 5A, the autonomous claim edit service 332 communicates with the records management system 354, the claim error processing service 350, the claim validation service 324, and one or more users 340 through one or more UIs (such as UIs 336a, . . . , 336k described above). In an example, communication between the autonomous claim edit service 332 and the records management system 354 may be through a storage repository 504. For example, the records management system 354 may store data within the storage repository 504, from where the autonomous claim edit service 332 may retrieve data.

As illustrated in FIG. 5A, the user 340 is the biller, a coder, or another user using the autonomous claim edit service 332. The user 340 is responsible for resolving errors or anomalies within the claims generated by the claim generation service 316 and flagged as being erroneous by the claim validation service 324. The autonomous claim edit service 332 (also referred to as a billing exception resolution application) enables resolution of billing exceptions, e.g., by resolving erroneous claims.

The records management system 354 interacts with the autonomous claim edit service 332, and includes one or more systems for managing medical documents and records pertinent to a patient encounter. Some example systems within the records management system 354 are illustrated in FIG. 5A, such as a supply chain information system 508, a clinical notes system 512, a surgical information system 516, a charge posting system 520, and/or one or more other systems managing medical records that may be used for generating and managing charges and medical documents, assigning codes, and/or generating claims.

In an example, the supply chain information system 508 provides supply chain information associated with equipment, medical devices, etc. used for medical procedures (such as surgeries), for which the patient and/or the insurance carrier is to be billed. For example, the supply chain information includes supply record of medical devices, involves for such medical devices, etc. The supply record and/or the invoice illustrated in the above-described UIs 336f and 336 of FIGS. 3F and 3G, respectively, may be maintained by the supply chain information system 508, in an example.

In an example, the clinical notes system 512 of the records management system 354 maintains notes and documents entered by healthcare providers (such as physicians, surgeons, nurses, medical technician, etc.) prior to, during, and/or subsequent to a patient encounter. The clinical record illustrated in the above-described UI 336e of FIG. 3E may be maintained by the clinical notes system 512, in an example.

In an example, the surgical information system 516 of the records management system 354 maintains notes and documents entered by healthcare providers (such as physicians, surgeons, nurses, medical technician, etc.) prior to, during, and/or subsequent to a patient encounter that involves a surgery or a medical procedure, such as periop records, preoperative records, postoperative records, etc. The periop record illustrated in the above-described UI 336d of FIG. 3D may be maintained by the surgical information system 516, in an example.

In an example, the charge posting system 520 of the records management system 354 manages financial data entered after a patient encounter, such as manages storage of charges, codes, claims, etc. of the medical coding and billing system 300.

FIGS. 5A and 5B also illustrate the claim error processing service 350 that works in conjunction with the autonomous claim edit service 332 for editing the claims, as described above with respect to UIs 336a-336k of FIGS. 4A-4K, respectively. In an example, the claim error processing service 350 comprises a ML model 552. The ML model 552 may be a generative AI service model, such as a large language model (LLM), or another type of generative AI service model. The claim error processing service 350 further comprises a feature extraction engine 551, as described below in detail. In an example, the claim error processing service 350 summarizes and provides insights from the records provided by the records management system 354, to assist in resolution of errors in claims, as also described below.

The storage repository 504 is used to store various records, information, and/or data (such as charges, codes, claims, etc.) of the medical coding and billing system 300.

Interactions between various components in FIG. 5A are indicated by a corresponding numbers 1, 2, 3, 4, and 5. These numbers indicate an example sequence of operation, such as operation 1 followed by operation 2, followed by operation 3, and so on. Although the operations are illustrated in a particular sequence in the example of FIG. 5A, the sequence may be different in other examples.

Initially (indicated by 1 within a circle in FIG. 5A), the user 340 interacts with the autonomous claim edit service 332, to request resolution of a claim being flagged as being erroneous, as illustrated in the UI 336b of FIG. 4B.

Subsequently, the autonomous claim edit service 332 makes API calls to the records management system 354, e.g., to retrieve relevant records, documents, and information associated with the erroneous claim currently being considered for resolution. The autonomous claim edit service 332 also interacts with the storage repository 504, to retrieve relevant records 550. The API call and the retrieval of the records 550 from the storage repository 504 are indicated by 2 within a circle in FIG. 5A.

Subsequently, the autonomous claim edit service 332 calls the claim error processing service 350 to perform review of the retrieved records 550, and transmits the retrieved records 550 to the claim error processing service 350 (indicated by 3 within a circle in FIG. 5A). The retrieved records 550 pertains to a patient encounter associated with the erroneous claim. Examples of such records 550 include medical documents pertinent to the patent encounter, such as one or more of (i) supply chain records including information associated with equipment, medical devices, etc. used for medical procedures (such as surgeries), for which the patient and/or the insurance carrier is to be billed, (ii) notes and documents entered by healthcare providers (such as physicians, surgeons, nurses, medical technician, etc.) prior to, during, and/or subsequent to the patient encounter, (iii) notes and documents entered by healthcare providers (such as physicians, surgeons, nurses, medical technician, etc.) prior to, during, and/or subsequent to a patient encounter that involves a surgery or a medical procedure, such as periop records, preoperative records, postoperative records, and/or any other relevant records associated with the patient encounter.

The records 550 are heterogeneous electronic medical records associated with the patient encounter. In an example, the records 550 includes one or more of structured billing codes, structured data, semi-structured data, and free-text clinical notes. In an example, structured data may comprise patient demographics, location of the patient encounter, name and designation of healthcare providers providing service during the patient encounter, etc. In an example, semi-structured data may comprise description of medical devices, surgical instruments and/or implants used during the patient encounter and/or a surgery, etc. In an example, free-text clinical notes may comprise any clinical notes entered by one or more healthcare professions during the patient encounter.

In an example, the claim error processing service 350 comprises the feature extraction engine 551, which processes the records 550. For example, feature extraction engine 551 transforms the plurality of heterogeneous electronic medical records 550 into a unified machine-readable representation comprising semantic embeddings 558 derived from one or more of the structured data, the semi-structured data, and the free-text clinical notes. For example, the feature extraction engine 551 acts as a unifying layer on the structured data, semi-structured data, and free-text clinical notes, and/or normalizes and represents the heterogeneous records 550 in a format that the ML model 552 can consume. For example, the feature extraction engine 551 may process structured data, such as perform one or more of one-hot encoding, normalization, vectorization of numeric and categorical features, map domain-specific values into embeddings (e.g., ICD-10 medical codes to embeddings), and/or combinations thereof. In an example, the feature extraction engine 551 may process semi-structured data, such as perform one or more of flatten and/or parse semi-structured data into key-value pairs, serialize into a canonical text form (e.g., “age: 42; medication: aspirin”), encode the schema (e.g., to preserve relationships), and/or combinations thereof. In an example, the feature extraction engine 551 may process free-text clinical notes, such as perform one or more of tokenize and feed into a pretrained embedding model. Thus, the feature extraction engine 551 transforms the structured billing codes, the structured data, the semi-structured data, and the free-text clinical notes of the records 550 into the unified machine-readable representation comprising semantic embeddings 558. The ML model(s) 552 receives the semantic embeddings 558, and error conditions 555 from the autonomous claim edit service 332. The error conditions 555 may be specified in the error report received by the autonomous claim edit service 332.

In an example, the ML model 552 analyzes the records 550 and/or the corresponding semantic embeddings 558 in view of the error condition 555. For example, the ML model 552 generates a mapping between the error condition 555 and one or more spans within the unified representation of the semantic embeddings 558. In an example, the mapping may be determined by the ML model 552 using (i) learned associations between claim error types and record modalities and/or (ii) the semantic embeddings. For example, the records 550 are associated with a plurality of record modalities. For example, the records 550 comprises one or more of supply chain records, clinical notes, periop records, preoperative records, postoperative records, and/or any other relevant records associated with the patient encounter.

The ML model 552 may be trained to look at a specific record modality in response to the error condition 555, e.g., based on a type of the error condition 555. For example, if the error condition is associated with a missing implant charges (e.g., described above with respect to FIGS. 4A-4K), then the ML model 552 may parse and analyze supply chain records and periop records, to find documentary evidence 560 implicitly and/or explicitly supporting use of the implant during the patient encounter. In another example, if the error condition is associated with a missing or erroneous modifier to a charge, the ML model 552 may review the patient after-visit summary, to identify a modifier to an evaluation and management service charge. In another example, if the error condition is associated with a missing location of service provided to a patient (such as whether the visit was an inpatient visit, or an outpatient visit), the ML model 552 reviews the patient after-visit summary, clinician notes, registration record, and/or other relevant records to identify the location of service.

In an example, the ML model 552 generates the mapping between the error condition and one or more spans within the unified representation, and based on such a mapping, identifies documentary evidence 560 within the one or more spans that satisfies a model-learned evidentiary relevance condition. In the above example where the error condition is associated with the missing implant charges, the ML model 552 identifies documentary evidence 560 within the supply chain records and periop records, which implicitly and/or explicitly supports use of the implant during the patient encounter. In another example, if the error condition is associated with a missing or erroneous modifier to a charge, the ML model 552 identifies documentary evidence 560 within the patient after-visit summary, which supports use of a correct modifier. In another example, if the error condition is associated with a missing location of service provided to a patient (such as whether the visit was an inpatient visit, or an outpatient visit), the ML model 552 identifies documentary evidence 560 within the patient after-visit summary, clinician notes, registration record, and/or other relevant records, which specifies the correct location of service.

Thus, the ML model 552 identifies and extracts most relevant information from the various records, and in view of the error condition 555 of the erroneous claim. Based on historical data and current information, the ML model 552 may suggest corrective automated actions for potential resolution of the erroneous claim. For example, the ML model 552 generates one or more machine-formatted corrective actions to resolve the error condition 555 in the claim, wherein each corrective action comprises structured data conforming to a claim submission standard and linked to the identified documentary evidence 560. Thus, a corrective action, if implemented, would conform the corrected claim to the claim submission standard. Moreover, a corrective action is linked to the identified documentary evidence 560 within the records 550.

For example, the ML model 552 may tailor the suggested corrective actions based on a nature of the error. For example, for the above-described example (e.g., described above with respect to FIGS. 4A-4K) where implant charges are missing, the ML model 552 identifies a plurality of records that implicitly and/or explicitly indicate usage of the implant during the surgery, so that suggested automated actions (see UI 336c) may include generating implant charges. The ML model 552 identifies documentary evidence 560 within the retrieved records, to support the suggested automated actions.

In another example, if an error for a claim shows a missing or erroneous modified to a charge, the ML model 552 reviews the patient after-visit summary and suggests an automated action to append a modifier to an evaluation and management service charge.

In yet another example, if an error for a claim shows a missing location of service provided to a patient (such as whether the visit was an inpatient visit, or an outpatient visit), the ML model 552 reviews the patient after-visit summary, clinician notes, registration record, and/or other relevant records to identify the location of service, and accordingly suggests adding the location of service to the claim. FIG. 2 described above illustrates example automated actions suggested by the ML model 552.

In an example, the ML model 552 outputs (i) the one or more machine-formatted corrective actions 564, and (ii) retrievable excerpts of the identified documentary evidence 560. After the ML model 552 outputs automated actions and the user agrees to such automated actions, the claim is corrected (e.g., as described above with respect to the examples of FIGS. 4A-4K). For example, the autonomous claim edit service 332 receives user input indicative of an acceptance of at least one of the machine-formatted corrective actions, and in response to the input, automatically applies the at least one of the machine-formatted corrective actions to the electronic claim, to generate the corrected claim.

The corrected claim is transmitted to the claim validation service 324, for revalidation of the corrected claim. If the claim validation service 324 successfully validates the claims, the corrected claim is now ready for submission to the insurance carrier. Communication between the autonomous claim edit service 332 and the claim validation service 324 are indicated by 4 within a circle in FIG. 5A.

After the corrected claim has been validated, the corrected claim is transmitted to the charge posting system 520. In an example, the claim filing service 328 described above with respect to FIG. 3 is a part of the charge posting system 520. The corrected claim is then filed, e.g., submitted to the medical insurance carrier for reimbursement.

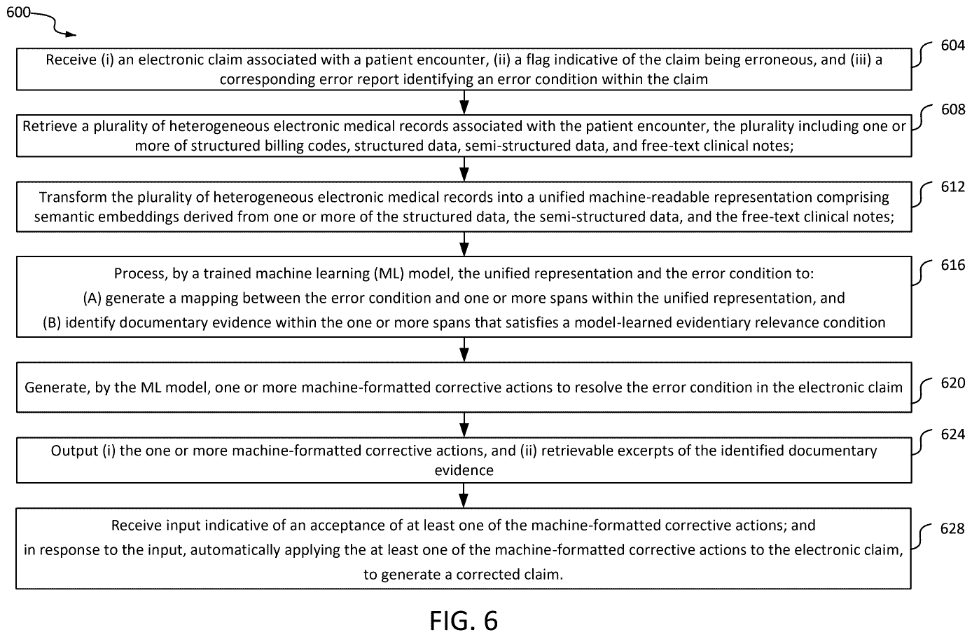

FIG. 6 illustrates a method 600 for autonomous editing of medical claims, in accordance with some examples. The method 600 can be implemented within the medical coding and billing system 300 of FIG. 3, and using the autonomous claim edit service 332 and/or the claim error processing service 350 of FIGS. 3 and 5A, 5B, in an example.

At 604, an electronic claim associated with a patient encounter is received, along with a flag indicative of the claim being erroneous, and a corresponding error report identifying an error condition within the claim. In an example, the claim is indicative of charges for a patient encounter. In an example, the error condition includes a reason behind the claim being flagged as erroneous.

At 608, a plurality of heterogeneous electronic medical records associated with the patient encounter are retrieved (e.g., based on communication between the autonomous claim edit service 332 and the records management system 354). In an example, the records include one or more of structured billing codes, structured data, semi-structured data, and free-text clinical notes.

At 612, the claim error processing service 350 (such as the feature extraction engine 551) transforms the plurality of heterogeneous electronic medical records into a unified machine-readable representation comprising semantic embeddings derived from one or more of the structured data, the semi-structured data, and the free-text clinical notes, as described above in further detail.

At 616, a trained machine learning (ML) model processes the unified representation and the error condition to generate a mapping between the error condition and one or more spans within the unified representation. In an example, the mapping is determined using (i) learned associations between claim error types and record modalities and/or (ii) the semantic embeddings. The ML model further identifies documentary evidence within the one or more spans that satisfies a model-learned evidentiary relevance condition, as described above in further detail.

At 620, the ML model generates one or more machine-formatted corrective actions to resolve the error condition in the electronic claim. In an example, each corrective action comprising structured data conforming to a claim submission standard and linked to the identified documentary evidence.

At 624, (i) the one or more machine-formatted corrective actions, and (ii) retrievable excerpts of the identified documentary evidence are output (e.g., by the autonomous claim edit service 332), such as displayed on a display screen that is viewable by a biller and/or a coder.

At 628, input indicative of an acceptance of at least one of the machine-formatted corrective actions is received (e.g., from a coder or biller, and by the autonomous claim edit service 332). In an example, in response to the input, the at least one of the machine-formatted corrective actions are automatically applied to the electronic claim, to generate a corrected claim (e.g., by the autonomous claim edit service 332).

### Computer System Architecture

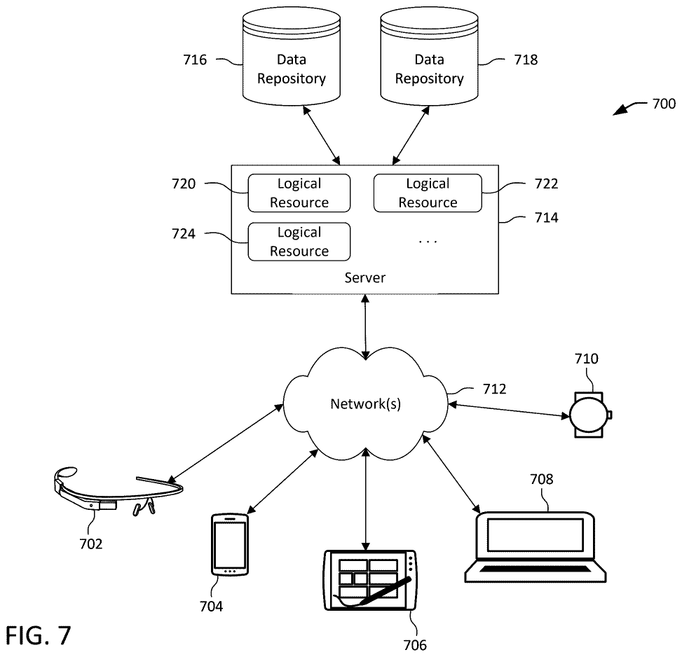

FIG. 7 depicts a simplified diagram of a distributed system 700 for implementing an embodiment. In the illustrated embodiment, distributed system 700 includes one or more client computing devices 702, 704, 706, 708, and/or 710 coupled to a server 714 via one or more communication networks 712. Clients computing devices 702, 704, 706, 708, and/or 710 may be configured to execute one or more applications.

In various aspects, server 714 may be adapted to run one or more services or software applications that enable techniques for autonomous editing of medical claims.

In certain aspects, server 714 may also provide other services or software applications that can include non-virtual and virtual environments. In some aspects, these services may be offered as web-based or cloud services, such as under a Software as a Service (SaaS) model to the users of client computing devices 702, 704, 706, 708, and/or 710. Users operating client computing devices 702, 704, 706, 708, and/or 710 may in turn utilize one or more client applications to interact with server 714 to utilize the services provided by these components.

In the configuration depicted in FIG. 7, server 714 may include one or more components 720, 722 and 724 that implement the functions performed by server 714. These components may include software components that may be executed by one or more processors, hardware components, or combinations thereof. It should be appreciated that various different system configurations are possible, which may be different from distributed system 700. The embodiment shown in FIG. 7 is thus one example of a distributed system for implementing an embodiment system and is not intended to be limiting.

Users may use client computing devices 702, 704, 706, 708, and/or 710 for techniques for autonomous editing of medical claims in accordance with the teachings of this disclosure. A client device may provide an interface that enables a user of the client device to interact with the client device. The client device may also output information to the user via this interface. Although FIG. 7 depicts only five client computing devices, any number of client computing devices may be supported.

The client devices may include various types of computing systems such as smart phones or other portable handheld devices, general purpose computers such as personal computers and laptops, workstation computers, personal assistant devices, smart watches, smart glasses, or other wearable devices, equipment firmware, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux® or Linux-like operating systems such as Oracle® Linux and Google Chrome® OS) including various mobile operating systems (e.g., Microsoft Windows Mobile®, iOS®, Windows Phone®, Android®, HarmonyOS®, Tizen®, KaiOS®, Sailfish® OS, Ubuntu® Touch, CalyxOS®). Portable handheld devices may include cellular phones, smartphones, (e.g., an iPhone®), tablets (e.g., iPad®), and the like. Virtual personal assistants such as Amazon® Alexa®, Google® Assistant, Microsoft® Cortana®, Apple® Siri®, and others may be implemented on devices with a microphone and/or camera to receive user or environmental inputs, as well as a speaker and/or display to respond to the inputs. Wearable devices may include Apple® Watch, Samsung Galaxy® Watch, Meta Quest®, Ray-Ban® Meta® smart glasses, Snap® Spectacles, and other devices. Gaming systems may include various handheld gaming devices, Internet-enabled gaming devices (e.g., a Microsoft Xbox® gaming console with or without a Kinect® gesture input device, Sony PlayStation® system, Nintendo Switch®, and other devices), and the like. The client devices may be capable of executing various different applications such as various Internet-related apps, communication applications (e.g., email applications, short message service (SMS) applications) and may use various communication protocols.

Network(s) 712 may be any type of network familiar to those skilled in the art that can support data communications using any of a variety of available protocols, including without limitation TCP/IP (transmission control protocol/Internet protocol), SNA (systems network architecture), IPX (Internet packet exchange), AppleTalk®, and the like. Merely by way of example, network(s) 712 can be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Server 714 may be composed of one or more general purpose computers, specialized server computers (including, by way of example, PC (personal computer) servers, UNIX® servers, LINIX® servers, mid-range servers, mainframe computers, rack-mounted servers, etc.), server farms, server clusters, a Real Application Cluster (RAC), database servers, or any other appropriate arrangement and/or combination. Server 714 can include one or more virtual machines running virtual operating systems, or other computing architectures involving virtualization such as one or more flexible pools of logical storage devices that can be virtualized to maintain virtual storage devices for the server. In various aspects, server 714 may be adapted to run one or more services or software applications that provide the functionality described in the foregoing disclosure.

The computing systems in server 714 may run one or more operating systems including any of those discussed above, as well as any commercially available server operating system. Server 714 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, SAP®, Amazon®, Sybase®, IBM® (International Business Machines), and the like.

In some implementations, server 714 may include one or more applications to analyze and consolidate data feeds and/or event updates received from users of client computing devices 702, 704, 706, 708, and/or 710. As an example, data feeds and/or event updates may include, but are not limited to, blog feeds, Threads® feeds, Twitter® feeds, Facebook® updates or real-time updates received from one or more third party information sources and continuous data streams, which may include real-time events related to sensor data applications, financial tickers, network performance measuring tools (e.g., network monitoring and traffic management applications), clickstream analysis tools, automobile traffic monitoring, and the like. Server 714 may also include one or more applications to display the data feeds and/or real-time events via one or more display devices of client computing devices 702, 704, 706, 708, and/or 710.

Distributed system 700 may also include one or more data repositories 716, 718. These data repositories may be used to store data and other information in certain aspects. For example, one or more of the data repositories 716, 718 may be used to store information for techniques for autonomous editing of medical claims. Data repositories 716, 718 may reside in a variety of locations. For example, a data repository used by server 714 may be local to server 714 or may be remote from server 714 and in communication with server 714 via a network-based or dedicated connection. Data repositories 716, 718 may be of different types. In certain aspects, a data repository used by server 714 may be a database, for example, a relational database, a container database, an Exadata® storage device, or other data storage and retrieval tool such as databases provided by Oracle Corporation® and other vendors. One or more of these databases may be adapted to enable storage, update, and retrieval of data to and from the database in response to structured query language (SQL)-formatted commands.

In certain aspects, one or more of data repositories 716, 718 may also be used by applications to store application data. The data repositories used by applications may be of different types such as, for example, a key-value store repository, an object store repository, or a general storage repository supported by a file system.

In one embodiment, server 714 is part of a cloud-based system environment in which various services may be offered as cloud services, for a single tenant or for multiple tenants where data, requests, and other information specific to the tenant are kept private from each tenant. In the cloud-based system environment, multiple servers may communicate with each other to perform the work requested by client devices from the same or multiple tenants. The servers communicate on a cloud-side network that is not accessible to the client devices in order to perform the requested services and keep tenant data confidential from other tenants.

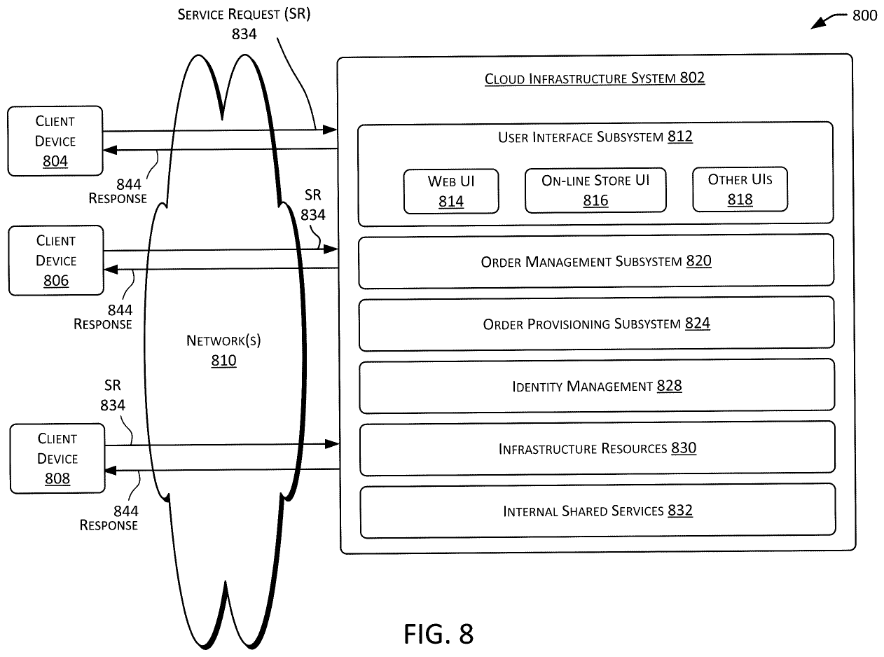

FIG. 8 is a simplified block diagram of a cloud-based system environment in which autonomous editing of medical claims are performed, in accordance with certain aspects. In the embodiment depicted in FIG. 8, cloud infrastructure system 802 may provide one or more cloud services that may be requested by users using one or more client computing devices 804, 806, and 808. Cloud infrastructure system 802 may comprise one or more computers and/or servers that may include those described above for server 714. The computers in cloud infrastructure system 802 may be organized as general purpose computers, specialized server computers, server farms, server clusters, or any other appropriate arrangement and/or combination.

Network(s) 810 may facilitate communication and exchange of data between clients 804, 806, and 808 and cloud infrastructure system 802. Network(s) 810 may include one or more networks. The networks may be of the same or different types. Network(s) 810 may support one or more communication protocols, including wired and/or wireless protocols, for facilitating the communications.

The embodiment depicted in FIG. 8 is only one example of a cloud infrastructure system and is not intended to be limiting. It should be appreciated that, in some other aspects, cloud infrastructure system 802 may have more or fewer components than those depicted in FIG. 8, may combine two or more components, or may have a different configuration or arrangement of components. For example, although FIG. 8 depicts three client computing devices, any number of client computing devices may be supported in alternative aspects.

The term cloud service is generally used to refer to a service that is made available to users on demand and via a communication network such as the Internet by systems (e.g., cloud infrastructure system 802) of a service provider. Typically, in a public cloud environment, servers and systems that make up the cloud service provider's system are different from the cloud customer's (“tenant's”) own on-premise servers and systems. The cloud service provider's systems are managed by the cloud service provider. Tenants can thus avail themselves of cloud services provided by a cloud service provider without having to purchase separate licenses, support, or hardware and software resources for the services. For example, a cloud service provider's system may host an application, and a user may, via a network 810 (e.g., the Internet), on demand, order and use the application without the user having to buy infrastructure resources for executing the application. Cloud services are designed to provide easy, scalable access to applications, resources, and services. Several providers offer cloud services. For example, several cloud services are offered by Oracle Corporation®, such as database services, middleware services, application services, and others.

In certain aspects, cloud infrastructure system 802 may provide one or more cloud services using different models such as under a Software as a Service (SaaS) model, a Platform as a Service (PaaS) model, an Infrastructure as a Service (IaaS) model, a Data as a Service (DaaS) model, and others, including hybrid service models. Cloud infrastructure system 802 may include a suite of databases, middleware, applications, and/or other resources that enable provision of the various cloud services.

A SaaS model enables an application or software to be delivered to a tenant's client device over a communication network like the Internet, as a service, without the tenant having to buy the hardware or software for the underlying application. For example, a SaaS model may be used to provide tenants access to on-demand applications that are hosted by cloud infrastructure system 802. Examples of SaaS services provided by Oracle Corporation® include, without limitation, various services for human resources/capital management, client relationship management (CRM), enterprise resource planning (ERP), supply chain management (SCM), enterprise performance management (EPM), analytics services, social applications, and others.

An IaaS model is generally used to provide infrastructure resources (e.g., servers, storage, hardware, and networking resources) to a tenant as a cloud service to provide elastic compute and storage capabilities. Various IaaS services are provided by Oracle Corporation®.

A PaaS model is generally used to provide, as a service, platform and environment resources that enable tenants to develop, run, and manage applications and services without the tenant having to procure, build, or maintain such resources. Examples of PaaS services provided by Oracle Corporation® include, without limitation, Oracle Database Cloud Service (DBCS), Oracle Java Cloud Service (JCS), data management cloud service, various application development solutions services, and others.

A DaaS model is generally used to provide data as a service. Datasets may searched, combined, summarized, and downloaded or placed into use between applications. For example, user profile data may be updated by one application and provided to another application. As another example, summaries of user profile information generated based on a dataset may be used to enrich another dataset.

Cloud services are generally provided on an on-demand self-service basis, subscription-based, elastically scalable, reliable, highly available, and secure manner. For example, a tenant, via a subscription order, may order one or more services provided by cloud infrastructure system 802. Cloud infrastructure system 802 then performs processing to provide the services requested in the tenant's subscription order. Cloud infrastructure system 802 may be configured to provide one or even multiple cloud services.

Cloud infrastructure system 802 may provide the cloud services via different deployment models. In a public cloud model, cloud infrastructure system 802 may be owned by a third party cloud services provider and the cloud services are offered to any general public tenant, where the tenant can be an individual or an enterprise. In certain other aspects, under a private cloud model, cloud infrastructure system 802 may be operated within an organization (e.g., within an enterprise organization) and services provided to clients that are within the organization. For example, the clients may be various departments or employees or other individuals of departments of an enterprise such as the Human Resources department, the Payroll department, etc., or other individuals of the enterprise. In certain other aspects, under a community cloud model, the cloud infrastructure system 802 and the services provided may be shared by several organizations in a related community. Various other models such as hybrids of the above mentioned models may also be used.

Client computing devices 804, 806, and 808 may be of different types (such as devices 702, 704, 706, and 708 depicted in FIG. 7) and may be capable of operating one or more client applications. A user may use a client device to interact with cloud infrastructure system 802, such as to request a service provided by cloud infrastructure system 802.

In some aspects, the processing performed by cloud infrastructure system 802 for providing chatbot services may involve big data analysis. This analysis may involve using, analyzing, and manipulating large data sets to detect and visualize various trends, behaviors, relationships, etc. within the data. This analysis may be performed by one or more processors, possibly processing the data in parallel, performing simulations using the data, and the like. For example, big data analysis may be performed by cloud infrastructure system 802 for determining the intent of an utterance. The data used for this analysis may include structured data (e.g., data stored in a database or structured according to a structured model) and/or unstructured data (e.g., data blobs (binary large objects)).

As depicted in the embodiment in FIG. 8, cloud infrastructure system 802 may include infrastructure resources 830 that are utilized for facilitating the provision of various cloud services offered by cloud infrastructure system 802. Infrastructure resources 830 may include, for example, processing resources, storage or memory resources, networking resources, and the like.

In certain aspects, to facilitate efficient provisioning of these resources for supporting the various cloud services provided by cloud infrastructure system 802 for different tenants, the resources may be bundled into sets of resources or resource modules (also referred to as “pods”). Each resource module or pod may comprise a pre-integrated and optimized combination of resources of one or more types. In certain aspects, different pods may be pre-provisioned for different types of cloud services. For example, a first set of pods may be provisioned for a database service, a second set of pods, which may include a different combination of resources than a pod in the first set of pods, may be provisioned for Java service, and the like. For some services, the resources allocated for provisioning the services may be shared between the services.

Cloud infrastructure system 802 may itself internally use services 832 that are shared by different components of cloud infrastructure system 802 and which facilitate the provisioning of services by cloud infrastructure system 802. These internal shared services may include, without limitation, a security and identity service, an integration service, an enterprise repository service, an enterprise manager service, a virus scanning and whitelist service, a high availability, backup and recovery service, service for enabling cloud support, an email service, a notification service, a file transfer service, and the like.

Cloud infrastructure system 802 may comprise multiple subsystems. These subsystems may be implemented in software, or hardware, or combinations thereof. As depicted in FIG. 8, the subsystems may include a user interface subsystem 812 that enables users of cloud infrastructure system 802 to interact with cloud infrastructure system 802. User interface subsystem 812 may include various different interfaces such as a web interface 814, an online store interface 816 where cloud services provided by cloud infrastructure system 802 are advertised and are purchasable by a consumer, and other interfaces 818. For example, a tenant may, using a client device, request (service request 834) one or more services provided by cloud infrastructure system 802 using one or more of interfaces 814, 816, and 818. For example, a tenant may access the online store, browse cloud services offered by cloud infrastructure system 802, and place a subscription order for one or more services offered by cloud infrastructure system 802 that the tenant wishes to subscribe to. The service request may include information identifying the tenant and one or more services that the tenant desires to subscribe to. For example, a tenant may place a subscription order for a chatbot related service offered by cloud infrastructure system 802. As part of the order, the client may provide information identifying the input (e.g. utterances).

In certain aspects, such as the embodiment depicted in FIG. 8, cloud infrastructure system 802 may comprise an order management subsystem (OMS) 820 that is configured to process the new order. As part of this processing, OMS 820 may be configured to: create an account for the tenant, if not done already; receive billing and/or accounting information from the tenant that is to be used for billing the tenant for providing the requested service to the tenant; verify the tenant information; upon verification, book the order for the tenant; and orchestrate various workflows to prepare the order for provisioning.

Once properly validated, OMS 820 may then invoke the order provisioning subsystem (OPS) 824 that is configured to provision resources for the order including processing, memory, and networking resources. The provisioning may include allocating resources for the order and configuring the resources to facilitate the service requested by the tenant order. The manner in which resources are provisioned for an order and the type of the provisioned resources may depend upon the type of cloud service that has been ordered by the tenant. For example, according to one workflow, OPS 824 may be configured to determine the particular cloud service being requested and identify a number of pods that may have been pre-configured for that particular cloud service. The number of pods that are allocated for an order may depend upon the size/amount/level/scope of the requested service. For example, the number of pods to be allocated may be determined based upon the number of users to be supported by the service, the duration of time for which the service is being requested, and the like. The allocated pods may then be customized for the particular requesting tenant for providing the requested service.

Cloud infrastructure system 802 may send a response or notification 844 to the requesting tenant to indicate when the requested service is now ready for use. In some instances, information (e.g., a link) may be sent to the tenant that enables the tenant to start using and availing the benefits of the requested services.

Cloud infrastructure system 802 may provide services to multiple tenants. For each tenant, cloud infrastructure system 802 is responsible for managing information related to one or more subscription orders received from the tenant, maintaining tenant data related to the orders, and providing the requested services to the tenant or clients of the tenant. Cloud infrastructure system 802 may also collect usage statistics regarding a tenant's use of subscribed services. For example, statistics may be collected for the amount of storage used, the amount of data transferred, the number of users, and the amount of system up time and system down time, and the like. This usage information may be used to bill the tenant. Billing may be done, for example, on a monthly cycle.

Cloud infrastructure system 802 may provide services to multiple tenants in parallel. Cloud infrastructure system 802 may store information for these tenants, including possibly proprietary information. In certain aspects, cloud infrastructure system 802 comprises an identity management subsystem (IMS) 828 that is configured to manage tenant's information and provide the separation of the managed information such that information related to one tenant is not accessible by another tenant. IMS 828 may be configured to provide various security-related services such as identity services, such as information access management, authentication and authorization services, services for managing tenant identities and roles and related capabilities, and the like.

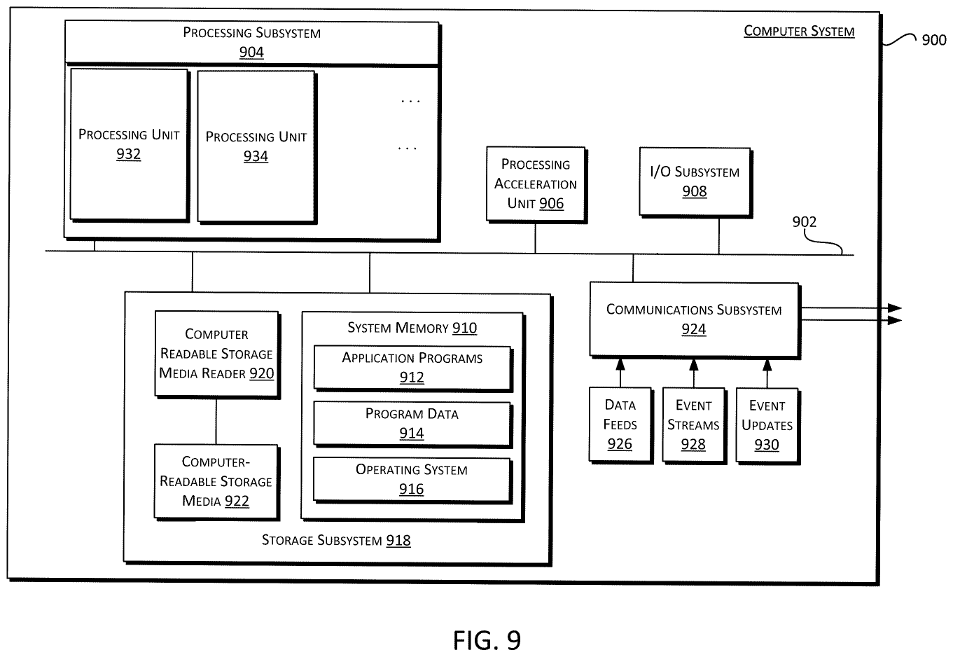

FIG. 9 illustrates an exemplary computer system 900 that may be used to implement certain aspects. As shown in FIG. 9, computer system 900 includes various subsystems including a processing subsystem 904 that communicates with a number of other subsystems via a bus subsystem 902. These other subsystems may include a processing acceleration unit 906, an I/O subsystem 908, a storage subsystem 918, and a communications subsystem 924. Storage subsystem 918 may include non-transitory computer-readable storage media including storage media 922 and a system memory 910.

Bus subsystem 902 provides a mechanism for letting the various components and subsystems of computer system 900 communicate with each other as intended. Although bus subsystem 902 is shown schematically as a single bus, alternative aspects of the bus subsystem may utilize multiple buses. Bus subsystem 902 may be any of several types of bus structures including a memory bus or memory controller, a peripheral bus, a local bus using any of a variety of bus architectures, and the like. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard, and the like.

Processing subsystem 904 controls the operation of computer system 900 and may comprise one or more processors, application specific integrated circuits (ASICs), or field programmable gate arrays (FPGAs). The processors may be single core or multicore processors. The processing resources of computer system 900 can be organized into one or more processing units 932, 934, etc. A processing unit may include one or more processors, one or more cores from the same or different processors, a combination of cores and processors, or other combinations of cores and processors. In some aspects, processing subsystem 904 can include one or more special purpose co-processors such as graphics processors, digital signal processors (DSPs), or the like. In some aspects, some or all of the processing units of processing subsystem 904 can be implemented using customized circuits, such as application specific integrated circuits (ASICs), or field programmable gate arrays (FPGAs).

In some aspects, the processing units in processing subsystem 904 can execute instructions stored in system memory 910 or on computer readable storage media 922. In various aspects, the processing units can execute a variety of programs or code instructions and can maintain multiple concurrently executing programs or processes. At any given time, some or all of the program code to be executed can be resident in system memory 910 and/or on computer-readable storage media 922 including potentially on one or more storage devices. Through suitable programming, processing subsystem 904 can provide various functionalities described above. In instances where computer system 900 is executing one or more virtual machines, one or more processing units may be allocated to each virtual machine.

In certain aspects, a processing acceleration unit 906 may optionally be provided for performing customized processing or for off-loading some of the processing performed by processing subsystem 904 so as to accelerate the overall processing performed by computer system 900.

I/O subsystem 908 may include devices and mechanisms for inputting information to computer system 900 and/or for outputting information from or via computer system 900. In general, use of the term input device is intended to include all possible types of devices and mechanisms for inputting information to computer system 900. User interface input devices may include, for example, a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may also include motion sensing and/or gesture recognition devices such as the Meta Quest® controller, Microsoft Kinect® motion sensor, the Microsoft Xbox® 360 game controller, or devices that provide an interface for receiving input using gestures and spoken commands. User interface input devices may also include eye gesture recognition devices such as a blink detector that detects eye activity (e.g., “blinking” while taking pictures and/or making a menu selection) from users and transforms the eye gestures as inputs to an input device. Additionally, user interface input devices may include voice recognition sensing devices that enable users to interact with voice recognition systems (e.g., Siri® navigator or Amazon Alexa®) through voice commands.

Other examples of user interface input devices include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, QR code readers, barcode readers, 3D scanners, 3D printers, laser rangefinders, and eye gaze tracking devices. Additionally, user interface input devices may include, for example, medical imaging input devices such as computed tomography, magnetic resonance imaging, position emission tomography, and medical ultrasonography devices. User interface input devices may also include, for example, audio input devices such as MIDI keyboards, digital musical instruments, and the like.

In general, use of the term output device is intended to include all possible types of devices and mechanisms for outputting information from computer system 900 to a user or other computer. User interface output devices may include a display subsystem, indicator lights, or non-visual displays such as audio output devices, etc. The display subsystem may be any device for outputting a digital picture. Example display devices include flat panel display devices such as those using a light emitting diode (LED) display, a liquid crystal display (LCD) or plasma display, a projection device, a touch screen, a desktop or laptop computer monitor, and the like. As another example, wearable display devices such as Meta Quest® or Microsoft HoloLens® may be mounted to the user for displaying information. User interface output devices may include, without limitation, a variety of display devices that visually convey text, graphics, and audio/video information such as monitors, printers, speakers, headphones, automotive navigation systems, plotters, voice output devices, and modems.

Storage subsystem 918 provides a repository or data store for storing information and data that is used by computer system 900. Storage subsystem 918 provides a tangible non-transitory computer-readable storage medium for storing the basic programming and data constructs that provide the functionality of some aspects. Storage subsystem 918 may store software (e.g., programs, code modules, instructions) that when executed by processing subsystem 904 provides the functionality described above. The software may be executed by one or more processing units of processing subsystem 904. Storage subsystem 918 may also provide a repository for storing data used in accordance with the teachings of this disclosure.

Storage subsystem 918 may include one or more non-transitory memory devices, including volatile and non-volatile memory devices. As shown in FIG. 9, storage subsystem 918 includes a system memory 910 and a computer-readable storage media 922. System memory 910 may include a number of memories including a volatile main random access memory (RAM) for storage of instructions and data during program execution and a non-volatile read only memory (ROM) or flash memory in which fixed instructions are stored. In some implementations, a basic input/output system (BIOS), containing the basic routines that help to transfer information between elements within computer system 900, such as during start-up, may typically be stored in the ROM. The RAM typically contains data and/or program modules that are presently being operated and executed by processing subsystem 904. In some implementations, system memory 910 may include multiple different types of memory, such as static random access memory (SRAM), dynamic random access memory (DRAM), and the like.

By way of example, and not limitation, as depicted in FIG. 9, system memory 910 may load application programs 912 that are being executed, which may include various applications such as Web browsers, mid-tier applications, relational database management systems (RDBMS), etc., program data 914, and an operating system 916. By way of example, operating system 916 may include various versions of Microsoft Windows®, Apple Macintosh®, and/or Linux® operating systems, a variety of commercially-available UNIX® or UNIX-like operating systems (including without limitation the variety of GNU/Linux operating systems, the Oracle Linux®, Google Chrome® OS, and the like) and/or mobile operating systems such as iOS, Windows® Phone, Android® OS, and others.

Computer-readable storage media 922 may store programming and data constructs that provide the functionality of some aspects. Computer-readable media 922 may provide storage of computer-readable instructions, data structures, program modules, and other data for computer system 900. Software (programs, code modules, instructions) that, when executed by processing subsystem 904 provides the functionality described above, may be stored in storage subsystem 918. By way of example, computer-readable storage media 922 may include non-volatile memory such as a hard disk drive, a magnetic disk drive, an optical disk drive such as a CD ROM, digital video disc (DVD), a Blu-Ray® disk, or other optical media. Computer-readable storage media 922 may include, but is not limited to, Zip® drives, flash memory cards, universal serial bus (USB) flash drives, secure digital (SD) cards, DVD disks, digital video tape, and the like. Computer-readable storage media 922 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, dynamic random access memory (DRAM)-based SSDs, magnetoresistive RAM (MRAM) SSDs, and hybrid SSDs that use a combination of DRAM and flash memory based SSDs.

In certain aspects, storage subsystem 918 may also include a computer-readable storage media reader 920 that can further be connected to computer-readable storage media 922. Reader 920 may receive and be configured to read data from a memory device such as a disk, a flash drive, etc.

In certain aspects, computer system 900 may support virtualization technologies, including but not limited to virtualization of processing and memory resources. For example, computer system 900 may provide support for executing one or more virtual machines. In certain aspects, computer system 900 may execute a program such as a hypervisor that facilitated the configuring and managing of the virtual machines. Each virtual machine may be allocated memory, compute (e.g., processors, cores), I/O, and networking resources. Each virtual machine generally runs independently of the other virtual machines. A virtual machine typically runs its own operating system, which may be the same as or different from the operating systems executed by other virtual machines executed by computer system 900. Accordingly, multiple operating systems may potentially be run concurrently by computer system 900.

Communications subsystem 924 provides an interface to other computer systems and networks. Communications subsystem 924 serves as an interface for receiving data from and transmitting data to other systems from computer system 900. For example, communications subsystem 924 may enable computer system 900 to establish a communication channel to one or more client devices via the Internet for receiving and sending information from and to the client devices. For example, the communications subsystem may be used to transmit a response to a user regarding the inquiry for a chatbot.

Communications subsystem 924 may support both wired and/or wireless communication protocols. For example, in certain aspects, communications subsystem 924 may include radio frequency (RF) transceiver components for accessing wireless voice and/or data networks (e.g., using cellular telephone technology, advanced data network technology, such as 3G, 4G or EDGE (enhanced data rates for global evolution), Wi-Fi (IEEE 802.XX family standards, or other mobile communication technologies, or any combination thereof), global positioning system (GPS) receiver components, and/or other components. In some aspects communications subsystem 924 can provide wired network connectivity (e.g., Ethernet) in addition to or instead of a wireless interface.

Communications subsystem 924 can receive and transmit data in various forms. For example, in some aspects, in addition to other forms, communications subsystem 924 may receive input communications in the form of structured and/or unstructured data feeds 926, event streams 928, event updates 930, and the like. For example, communications subsystem 924 may be configured to receive (or send) data feeds 926 in real-time from users of social media networks and/or other communication services such as Twitter® feeds, Facebook® updates, web feeds such as Rich Site Summary (RSS) feeds, and/or real-time updates from one or more third party information sources.

In certain aspects, communications subsystem 924 may be configured to receive data in the form of continuous data streams, which may include event streams 928 of real-time events and/or event updates 930, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 924 may also be configured to communicate data from computer system 900 to other computer systems or networks. The data may be communicated in various different forms such as structured and/or unstructured data feeds 926, event streams 928, event updates 930, and the like to one or more databases that may be in communication with one or more streaming data source computers coupled to computer system 900.

Computer system 900 can be one of various types, including a handheld portable device (e.g., an iPhone® cellular phone, an iPad® computing tablet, a personal digital assistant (PDA)), a wearable device (e.g., a Meta Quest® head mounted display), a personal computer, a workstation, a mainframe, a kiosk, a server rack, or any other data processing system. Due to the ever-changing nature of computers and networks, the description of computer system 900 depicted in FIG. 9 is intended only as a specific example. Many other configurations having more or fewer components than the system depicted in FIG. 9 are possible. Based on the disclosure and teachings provided herein, a person of ordinary skill in the art can appreciate other ways and/or methods to implement the various aspects.

Although specific aspects have been described, various modifications, alterations, alternative constructions, and equivalents are possible. Embodiments are not restricted to operation within certain specific data processing environments, but are free to operate within a plurality of data processing environments. Additionally, although certain aspects have been described using a particular series of transactions and steps, it should be apparent to those skilled in the art that this is not intended to be limiting. Although some flowcharts describe operations as a sequential process, many of the operations can be performed in parallel or concurrently. In addition, the order of the operations may be rearranged. A process may have additional steps not included in the figure. Various features and aspects of the above-described aspects may be used individually or jointly.

Further, while certain aspects have been described using a particular combination of hardware and software, it should be recognized that other combinations of hardware and software are also possible. Certain aspects may be implemented only in hardware, or only in software, or using combinations thereof. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques including but not limited to conventional techniques for inter-process communications, and different pairs of processes may use different techniques, or the same pair of processes may use different techniques at different times.

Specific details are given in this disclosure to provide a thorough understanding of the aspects. However, aspects may be practiced without these specific details. For example, well-known circuits, processes, algorithms, structures, and techniques have been shown without unnecessary detail in order to avoid obscuring the aspects. This description provides example aspects only, and is not intended to limit the scope, applicability, or configuration of other aspects. Rather, the preceding description of the aspects can provide those skilled in the art with an enabling description for implementing various aspects. Various changes may be made in the function and arrangement of elements.

The specification and drawings are, accordingly, to be regarded in an illustrative rather than a restrictive sense. It can, however, be evident that additions, subtractions, deletions, and other modifications and changes may be made thereunto without departing from the broader spirit and scope as set forth in the claims. Thus, although specific aspects have been described, these are not intended to be limiting. Various modifications and equivalents are within the scope of the following claims.

What is claimed is:

1. A computer-implemented method comprising:
receiving, by a computing system, (i) an electronic claim associated with a patient encounter, (ii) a flag indicative of the claim being erroneous, and (iii) a corresponding error report identifying an error condition within the claim;
retrieving, by the computing system, a plurality of heterogeneous electronic medical records associated with the patient encounter, the plurality including one or more of structured billing codes, structured data, semi-structured data, and free-text clinical notes;
transforming, by a feature extraction engine, the plurality of heterogeneous electronic medical records into a unified machine-readable representation comprising semantic embeddings derived from one or more of the structured data, the semi-structured data, and the free-text clinical notes;
processing, by a trained machine learning (ML) model, the unified representation and the error condition to:
generate a mapping between the error condition and one or more spans within the unified representation, the mapping determined using (i) learned associations between claim error types and record modalities and/or (ii) the semantic embeddings, and
identify documentary evidence within the one or more spans that satisfies a model-learned evidentiary relevance condition;
generating, by the ML model, one or more machine-formatted corrective actions to resolve the error condition in the electronic claim, each corrective action comprising structured data conforming to a claim submission standard and linked to the identified documentary evidence;
outputting (i) the one or more machine-formatted corrective actions, and (ii) retrievable excerpts of the identified documentary evidence;
receiving input indicative of an acceptance of at least one of the machine-formatted corrective actions; and
in response to the input, automatically applying the at least one of the machine-formatted corrective actions to the electronic claim, to generate a corrected claim.

2. The method of claim 1, further comprising:
validating the corrected claim; and
in response to validating the corrected claim, filing the corrected claim, to cause the claim to be submitted to an insurance carrier for reimbursement.

3. The method of claim 1, wherein the plurality of heterogeneous electronic medical records comprises one or more of (i) a supply chain record including information associated with medical devices used for a medical procedure for the patient encounter, (ii) a clinical note documenting the patient encounter, (iii) a surgical perioperative record, a surgical preoperative record, and/or a surgical postoperative record for the patient encounter, and (iv) a charge posting record including information associated with one or more charges, codes, and/or the claim associated with the patient encounter.

4. The method of claim 1, wherein the documentary evidence within the spans supports a corresponding corrective action to resolve the error condition.

5. The method of claim 1, wherein:
the claim is associated with a surgical procedure during the patient encounter;
the error condition is associated with a medical implant, which was used during the surgical procedure, missing in the claim;
retrieving the plurality of heterogeneous electronic medical records comprises retrieving one or more of (i) a perioperative patient record for the surgical procedure, (ii) a clinical record documenting the surgical procedure, (iii) supply record documenting a supply of the medical implant for the surgical procedure, and (iv) an invoice for the medical implant;
identifying the documentary evidence within the spans comprises identifying at least one of the plurality of retrieved heterogeneous electronic medical records that implicitly or explicitly supports use of the medical implant for the surgical procedure; and
the corrective actions include an action to add an indication of usage of the medical implant to the claim.

6. The method of claim 1, wherein:
the error condition indicates that the claim is missing an indication of a location where the patient encounter occurred;
retrieving the plurality of heterogeneous electronic medical records comprises retrieving one or more of medical records including the indication of the location where the patient encounter occurred;
identifying the documentary evidence within the spans comprises identifying at least one of the plurality of retrieved heterogeneous electronic medical records that includes the indication of the location where the patient encounter occurred; and
the corrective actions include adding the indication of the location where the patient encounter occurred to the claim.

7. The method of claim 1, wherein the ML model comprises a generative artificial intelligence (AI) model.

8. A non-transitory computer-readable medium including instructions that when executed by one or more processors, cause a system including the one or more processors to perform a set of operations including:
receiving, by a computing system, (i) an electronic claim associated with a patient encounter, (ii) a flag indicative of the claim being erroneous, and (iii) a corresponding error report identifying an error condition within the claim;
retrieving, by the computing system, a plurality of heterogeneous electronic medical records associated with the patient encounter, the plurality including one or more of structured billing codes, structured data, semi-structured data, and free-text clinical notes;
transforming, by a feature extraction engine, the plurality of heterogeneous electronic medical records into a unified machine-readable representation comprising semantic embeddings derived from one or more of the structured data, the semi-structured data, and the free-text clinical notes;
processing, by a trained machine learning (ML) model, the unified representation and the error condition to:
generate a mapping between the error condition and one or more spans within the unified representation, the mapping determined using (i) learned associations between claim error types and record modalities and/or (ii) the semantic embeddings, and
identify documentary evidence within the one or more spans that satisfies a model-learned evidentiary relevance condition;
generating, by the ML model, one or more machine-formatted corrective actions to resolve the error condition in the electronic claim, each corrective action comprising structured data conforming to a claim submission standard and linked to the identified documentary evidence;
outputting (i) the one or more machine-formatted corrective actions, and (ii) retrievable excerpts of the identified documentary evidence;
receiving input indicative of an acceptance of at least one of the machine-formatted corrective actions; and
in response to the input, automatically applying the at least one of the machine-formatted corrective actions to the electronic claim, to generate a corrected claim.

9. The non-transitory computer-readable medium of claim 8, wherein the set of operations include:
validating the corrected claim; and
in response to validating the corrected claim, filing the corrected claim, to cause the claim to be submitted to an insurance carrier for reimbursement.

10. The non-transitory computer-readable medium of claim 8, wherein the plurality of heterogeneous electronic medical records comprises one or more of (i) a supply chain record including information associated with medical devices used for a medical procedure for the patient encounter, (ii) a clinical note documenting the patient encounter, (iii) a surgical perioperative record, a surgical preoperative record, and/or a surgical postoperative record for the patient encounter, and (iv) a charge posting record including information associated with one or more charges, codes, and/or the claim associated with the patient encounter.

11. The non-transitory computer-readable medium of claim 8, wherein the documentary evidence within the spans supports a corresponding corrective action to resolve the error condition.

12. The non-transitory computer-readable medium of claim 8, wherein:
the claim is associated with a surgical procedure during the patient encounter;
the error condition is associated with a medical implant, which was used during the surgical procedure, missing in the claim;
retrieving the plurality of heterogeneous electronic medical records comprises retrieving one or more of (i) a perioperative patient record for the surgical procedure, (ii) a clinical record documenting the surgical procedure, (iii) supply record documenting a supply of the medical implant for the surgical procedure, and (iv) an invoice for the medical implant;
identifying the documentary evidence within the spans comprises identifying at least one of the plurality of retrieved heterogeneous electronic medical records that implicitly or explicitly supports use of the medical implant for the surgical procedure; and
the corrective actions include an action to add an indication of usage of the medical implant to the claim.

13. The non-transitory computer-readable medium of claim 8, wherein:
the error condition indicates that the claim is missing an indication of a location where the patient encounter occurred;
retrieving the plurality of heterogeneous electronic medical records comprises retrieving one or more of medical records including the indication of the location where the patient encounter occurred;
identifying the documentary evidence within the spans comprises identifying at least one of the plurality of retrieved heterogeneous electronic medical records that includes the indication of the location where the patient encounter occurred; and
the corrective actions include adding the indication of the location where the patient encounter occurred to the claim.

14. The non-transitory computer-readable medium of claim 8, wherein the ML model comprises a generative artificial intelligence (AI) model.

15. A system comprising:
one or more processors; and
one or more non-transitory computer-readable media storing instructions, which, when executed by the system, cause the system to perform a set of operations including:
receiving, by a computing system, (i) an electronic claim associated with a patient encounter, (ii) a flag indicative of the claim being erroneous, and (iii) a corresponding error report identifying an error condition within the claim;
retrieving, by the computing system, a plurality of heterogeneous electronic medical records associated with the patient encounter, the plurality including one or more of structured billing codes, structured data, semi-structured data, and free-text clinical notes;
transforming, by a feature extraction engine, the plurality of heterogeneous electronic medical records into a unified machine-readable representation comprising semantic embeddings derived from one or more of the structured data, the semi-structured data, and the free-text clinical notes;
processing, by a trained machine learning (ML) model, the unified representation and the error condition to:
generate a mapping between the error condition and one or more spans within the unified representation, the mapping determined using (i) learned associations between claim error types and record modalities and/or (ii) the semantic embeddings, and
identify documentary evidence within the one or more spans that satisfies a model-learned evidentiary relevance condition;
generating, by the ML model, one or more machine-formatted corrective actions to resolve the error condition in the electronic claim, each corrective action comprising structured data conforming to a claim submission standard and linked to the identified documentary evidence;
outputting (i) the one or more machine-formatted corrective actions, and (ii) retrievable excerpts of the identified documentary evidence;
receiving input indicative of an acceptance of at least one of the machine-formatted corrective actions; and
in response to the input, automatically applying the at least one of the machine-formatted corrective actions to the electronic claim, to generate a corrected claim.

16. The system of claim 15, wherein the set of operations include:
validating the corrected claim; and
in response to validating the corrected claim, filing the corrected claim, to cause the claim to be submitted to an insurance carrier for reimbursement.

17. The system of claim 15 wherein the plurality of heterogeneous electronic medical records comprises one or more of (i) a supply chain record including information associated with medical devices used for a medical procedure for the patient encounter, (ii) a clinical note documenting the patient encounter, (iii) a surgical perioperative record, a surgical preoperative record, and/or a surgical postoperative record for the patient encounter, and (iv) a charge posting record including information associated with one or more charges, codes, and/or the claim associated with the patient encounter.

18. The system of claim 15, wherein the documentary evidence within the spans supports a corresponding corrective action to resolve the error condition.

19. The system of claim 15, wherein:
the claim is associated with a surgical procedure during the patient encounter;
the error condition is associated with a medical implant, which was used during the surgical procedure, missing in the claim;
retrieving the plurality of heterogeneous electronic medical records comprises retrieving one or more of (i) a perioperative patient record for the surgical procedure, (ii) a clinical record documenting the surgical procedure, (iii) supply record documenting a supply of the medical implant for the surgical procedure, and (iv) an invoice for the medical implant;
identifying the documentary evidence within the spans comprises identifying at least one of the plurality of retrieved heterogeneous electronic medical records that implicitly or explicitly supports use of the medical implant for the surgical procedure; and
the corrective actions include an action to add an indication of usage of the medical implant to the claim.

20. The system of claim 15, wherein:
the error condition indicates that the claim is missing an indication of a location where the patient encounter occurred;
retrieving the plurality of heterogeneous electronic medical records comprises retrieving one or more of medical records including the indication of the location where the patient encounter occurred;
identifying the documentary evidence within the spans comprises identifying at least one of the plurality of retrieved heterogeneous electronic medical records that includes the indication of the location where the patient encounter occurred; and
the corrective actions include adding the indication of the location where the patient encounter occurred to the claim.
